(19) United States
(12) Patent Application Publication
(10) Pub. No.: US 2015/0177241 A1
(43) Pub. Date: Jun. 25, 2015

(54) BIOMARKERS OF IMMUNE RESPONSE IN MUCOSAL LESIONS AND THEIR USE WITH THERAPEUTIC VACCINATION

(71) Applicant: The Johns Hopkins University, Baltimore, MD (US)

(72) Inventor: Cornelia Trimble, Baltimore, MD (US)

(21) Appl. No.: 14/576,551

(22) Filed: Dec. 19, 2014

Related U.S. Application Data
(60) Provisional application No. 61/919,399, filed on Dec. 20, 2013.

(51) Int. Cl. G01N 33/569; C12N 7/00; A61K 39/12

(52) U.S. Cl. CPC G01N 33/56972; A61K 39/12; C12N 7/00; A61K 2039/585

(57) ABSTRACT

The present invention provides evidence of post-vaccination immunologic changes in target HPV induced cervical intraepithelial neoplasias that suggest the induction of clinically relevant tissue-localized immune responses, despite modest detectable responses in circulating T lymphocytes. The inventive methods allow for identification of immunological responses to HPV immunotherapies, and means for monitoring responses during clinical trials and treatment regimens.

### REFERENCE TO RELATED APPLICATIONS

This application claims the benefit of U.S. Provisional Patent Application No. 61/919,399, filed on Dec. 20, 2013, which is hereby incorporated by reference for all purposes as if fully set forth herein.

### STATEMENT OF GOVERNMENTAL INTEREST

This invention was made with U.S. Government support under grant nods. P50 CA098252, 1R21CA123876, R01 CA142691, and 5P30-CA6973 from the National Institutes of Health. The U.S. Government has certain rights in the invention.

### INCORPORATION-BY-REFERENCE OF MATERIAL SUBMITTED ELECTRONICALLY

The instant application contains a Sequence Listing which has been submitted in ASCII format via EFS-Web and is hereby incorporated by reference in its entirety. Said ASCII copy, created on Dec. 19, 2014, is named P12894-02_ST25.txt and is 3,101 bytes in size.

### BACKGROUND OF THE INVENTION

On a global scale, human papillomavirus (HPV), most commonly serotype 16 (HPV16), causes about 30% of cancers attributable to infectious pathogens (1). Persistent infection with an oncogenic HPV genotype is associated with subsequent cancers of the cervix, vagina, vulva, anus, and oropharynx [reviewed in (2)]. For many reasons, HPV infections are essentially endemic. Asymptomatic transmission occurs shortly after initiation of sexual intercourse (3). Infections are anatomically restricted to mucosal epithelium, do not cause grossly visible lesions, and do not elicit systemic symptoms. The clinically indolent nature of these infections facilitates maintenance of a large herd burden of transmissible HPV.

Despite the availability of screening methods and the introduction of preventative HPV vaccines, high-grade squamous intraepithelial neoplasia, the precursor to invasive squamous cancers, remains common. Rates of preventive vaccination in eligible U.S. cohorts, young people aged 9 to 26 years, have been suboptimal; in 2010, less than half of adolescent girls aged 13 to 17 years had initiated vaccination, and the rates of vaccination are decreasing (4). Moreover, the incidence of HPV-associated cancers in anatomic sites for which screening algorithms have not yet been identified, particularly in the oropharynx, is increasing steadily and will likely bypass that of cervical cancer in the near future (2). In the end, although much has been learned about the immunobiology of HPV-associated disease, the problem of translation of this knowledge into strategies to prevent and treat disease remains unresolved.

High-grade cervical intraepithelial neoplasia (CIN2/3) is a lesion that should be susceptible to an HPV-specific immune response. The development of cervical cancer and its precursor CIN lesions is associated with integration of the HPV genome into the host genome and subsequent expression of two HPV early gene products, E6 and E7, which inactivate p53 and pRb, respectively (5, 6). Expression of both of these viral, “non-self” proteins is functionally required to initiate and maintain the transformed phenotype, thereby providing true tumor-specific antigenic targets (5, 6).

Preinvasive, intraepithelial dysplastic lesions caused by HPV are clinically indolent. Whereas all cervical squamous carcinomas arise from untreated CIN2/3, not all CIN2/3 lesions progress to invasive cancer. We and others have reported that in a time frame of 4 to 6 months, ˜35% of CIN2/3 lesions undergo spontaneous regression (7, 8). Lesions resulting from mono-infection with HPV16 were less likely to undergo regression than those caused by other HPV genotypes: in this time frame, ˜20 to 25% of HPV16-associated CIN2/3 lesions regressed. Regardless of whether or not lesions regressed, T cell responses to HPV16 E6 and E7 were marginal, requiring expansion by ex vivo sensitization for detection (9, 10). The fact that neither the magnitude nor the breadth of naturally occurring T cell responses detected in the blood was a robust predictor of regression of preinvasive HPV disease of the cervix raised the question of whether immune cells in the lesional mucosa would be more informative.

Indeed, both the magnitude and distribution of CD8+ T cell infiltrates in dysplastic mucosa provided prognostic information. In persistent disease, CD8+ T cells were restricted to lesional stroma and failed to access the lesional epithelium. In contrast, dysplastic lesions that “permitted” CD8+ T cell access to the epithelial compartment were significantly more likely to undergo subsequent regression (11). These observations suggested that, although CD8 T cells are likely to play a role in elimination of preinvasive, incipient HPV-associated neoplasia, access to lesional epithelium presents a critical obstacle.

Previous attempts to translate therapeutic vaccination strategies for HPV disease have yielded limited success by two standard measures of vaccine efficacy: (i) induction of robust peripheral blood T cell responses to vaccine antigen and (ii) correlation of peripheral blood immune responses with histologic regression of disease. Here, we report evidence of post-vaccination immunologic changes in target lesions that suggest the induction of clinically relevant tissue-localized immune responses, despite modest detectable responses in circulating T lymphocytes. The identification of post-vaccination changes in target lesions has practical implications for the design and interpretation of immunotherapeutic trials for preinvasive HPV disease.

As such there still exists a need for methods of identification of novel and prognosticative biomarkers which are correlated with tissue-localized immune responses in HPV associated lesions.

### SUMMARY OF THE INVENTION

In accordance with an embodiment, the present invention provides a method for determining the effectiveness of an HPV vaccine regimen on a subject having a cervical intraepithelial neoplasia (CIN) comprising: a) obtaining at least one first tissue sample from the CIN of the subject; b) administering to the subject an effective amount of a first vaccine construct comprising HPV16 E7 antigen one or more times; c) administering to the subject an effective amount of a second vaccine construct comprising both E6 and E7 antigens from both HPV16 and HPV18; d) obtaining at least one or more second tissue samples from the CIN of the subject about 4 to about 24 weeks after step c); e)measuring the amount of CD8+ T cell infiltrates in the intraepithelial tissue of the first and second CIN samples; and f) determining that the HPV vaccine regimen is effective when the amount of CD8+ T cell infiltrates in the second tissue sample is increased when compared to the amount of CD8+ T cell infiltrates in the first tissue sample.

In accordance with an embodiment, the present invention provides a method for treating a subject having a cervical intraepithelial neoplasia (CIN) comprising: a) obtaining at least one first tissue sample from the CIN of the subject; b) administering to the subject an effective amount of a first vaccine construct comprising HPV16 E7 antigen one or more times; c) administering to the subject an effective amount of a second vaccine construct comprising both E6 and E7 antigens from both HPV16 and HPV18; d) obtaining at least one or more second tissue samples from the CIN of the subject about 4 to about 24 weeks after step c); e) measuring the amount of CD8+ T cell infiltrates in the intraepithelial tissue of the first and second CIN samples; and f) determining that the HPV treatment is effective when the amount of CD8+ T cell infiltrates in the second tissue sample is increased when compared to the amount of CD8+ T cell infiltrates in the first tissue sample.

In accordance with an embodiment, the present invention provides a method for determining the effectiveness of an HPV vaccine regimen with an adjuvant on a subject having a cervical intraepithelial neoplasia (CIN) comprising: a) obtaining at least one first tissue sample from the CIN of the subject; b) administering to the subject an effective amount of a first vaccine construct comprising HPV16 E7 antigen one or more times; c) administering to the subject an effective amount of a second vaccine construct comprising both E6 and E7 antigens from both HPV16 and HPV18, and an effective amount of an adjuvant; d) obtaining at least one or more second tissue samples from the CIN of the subject about 4 to about 24 weeks after step c); e) measuring the amount of CD8+ T cell infiltrates in the intraepithelial tissue of the first and second CIN samples; and f) determining that the HPV treatment is effective when the amount of CD8+ T cell infiltrates in the second tissue sample is increased when compared to the amount of CD8+ T cell infiltrates in the first tissue sample.

In accordance with an embodiment, the present invention provides a method for treating a subject having a cervical intraepithelial neoplasia (CIN) comprising: a) obtaining at least one first tissue sample from the CIN of the subject; b) administering to the subject an effective amount of a first vaccine construct comprising HPV16 E7 antigen one or more times; c) administering to the subject an effective amount of a second vaccine construct comprising both E6 and E7 antigens from both HPV16 and HPV18, and an effective amount of an adjuvant; d) obtaining at least one or more second tissue samples from the CIN of the subject about 4 to about 24 weeks after step c); e) measuring the quantity of CD8+ T c amount of CD8+ T cell infiltrates in the intraepithelial tissue of the first and second CIN samples; and f) determining that the HPV treatment is effective when the amount of CD8+ T cell infiltrates in the second tissue sample is increased when compared to the amount of CD8+ T cell infiltrates in the first tissue sample.

### BRIEF DESCRIPTION OF THE DRAWINGS

FIGS. 1A-1C show the intramuscular immunization with DNAE₇ prime, followed by recombinant vaccinia (rVac_{E6E7}, also known as TA-HPV) boost, induces HPV16 E6 and E7-specific T_{H}1 immune responses in the peripheral blood. (1A) Cellular immune responses in unfractionated PBMCs, quantified by IFN-_{γ} ELISpot assays. All subjects received two intramuscular DNA vaccinations at weeks 0 and 4. Dose escalation of recombinant vaccinia (rVac_{E6E7}) boost vaccination at week 8; subjects received either 1.6×10⁵ PFU (DDV1), 1.6×10⁶ PFU (DDV2), or 1.6×10⁷ PFU (DDV3). Results are expressed as SFUs per 10⁶ PBMCs. (1B and C) Multiparametric flow cytometry was used to detect HPV16-and HPV 18-specific production of IFN-γ. Bar graphs summarizing ICS and flow cytometry analyses of PBMC response to HPV16 and HPV18 peptides: (1B) CD8⁺IFN-γ⁺ and (1C) CD4⁺IFN-γ⁺. Subjects with complete histologic regression at week 15 are designated with a bar below the subject ID.

FIGS. 2A-2D depict tissue CD8+ T cell infiltrates in the target lesion increase after vaccination. (2A) Representative immunohistochemical (IHC) staining for CD8 in lesional tissue before (left column) and after (right column) vaccination (patient 3009). (2B) These infiltrates are Tbet+. (2C) In contrast, the intensity of Foxp3+ infiltrates does not change substantially. (2D) Bar graphs depicting quantitated CD8+ and Foxp3+ infiltrates, and the ratio of CD8/Foxp3+ cells in epithelium (e) and stroma (s) of CIN3, before and after vaccination, in all study subjects. Data from bar graphs are means of 3 to 10 regions of interest (ROIs) quantitated per tissue compartment per subject. Error bars show SEM. P<0.05, **P<0.01, Wilcoxon signed rank test. Scale bars, 50 82 m.

FIGS. 3A-3E show that in vaccinated patients, the cervix is infiltrated by activated effector memory T cells with potent effector functions. (3A to 3D) T cells were isolated from the cervix of healthy controls (normal, n=5), patients with untreated CIN2/3 (n =7), and patients with CIN2/3 after vaccination (n=4). The percent of CD4 versus CD8 T cells (3A), the CD4/CD8 ratio (3B), the percent Foxp3⁺ T_{reg} (3C), and the CD8/Foxp3⁺ ratio (3D) are shown. The CD4/CD8 ratio tended to increase in untreated CIN and normalize in vaccinated patients. The CD8/Foxp3⁺ ratio was lowest in untreated CIN and tended to increase after vaccination, although only the normal versus CIN2/3 group reached statistical significance. Data from bar graphs are means of cases analyzed. Error bars show SEM. P<0.05, Wilcoxon rank sum test. (3E) Representative histograms of surface phenotype and cytokine production of T cells from vaccinated patients (n=4). Surface phenotype stains were performed on unstimulated cells, and cytokine analysis stains were performed after stimulation with phorbol 12-myristate 13-acetate and ionomycin. Most of the T cells from patients after vaccination were memory CD45RO⁺ T cells, many expressed the activation marker CD69, most lacked the central memory markers L-selectin/CCR7, and all expressed the gut-tropic homing receptor α₄β₇ integrin. Most of both CD4 and CD8 T cells produced IFN-γ, and a significant subset of CD4 T cells expressed interleukin-17 (IL-17) and/or IL-22. All histograms are gated to show CD3⁺ T cells; the gating strategy and representative isotype controls are included in FIG. 9.

FIGS. 4A-4C depict post-vaccination lymphoid neogenesis in target lesions. (4A) Hematoxylin and eosin-stained sections demonstrate organized lymphoid structures that are localized to the stroma immediately subjacent to residual dysplastic epithelium (top row, ×64 magnification; second row, ×160 magnification). Inflammatory infiltrates are accompanied by high endothelial venule-like vessels (triangle points, black dotted outline), access to dysplastic epithelium (white dotted outline), and lesional epithelial apoptosis (fat arrows). (4B) TLSs in postvaccination stroma express Ki67, central CD20 (a pan-B cell marker), CD3 (a pan-T cell marker), and peripheral lymph node addressin (iPNAd), which identifies vascular endothelium in high endothelial venules. (4C) Representative IHC of Ki67 before (left) and after (right) vaccination, and bar graph summarizing quantitative image analysis of Ki67⁺ in lesional stroma before and after vaccination. Data from bar graphs are means of 3 to 10 ROIs quantitated per tissue compartment for each subject. Error bars show SEM. **P<0.01, Wilcoxon signed rank test. Scale bars, 50 μm.

FIGS. 5A-5B show that post-vaccination stromal TLSs are associated with a functional signature. Laser capture microdissection of cervical mucosal epithelium and subjacent stroma from normal (three), CIN2/3 (three), and vaccinated CIN2/3 (three) cases. (5A) In lesional epithelium overlying TLSs, expression of CD8 and CXCR3 is increased in postvaccination tissue. (5B) Transcripts for IFN-β are increased in the stromal compartment of vaccinated CIN2/3. Data from scatter plots are means of duplicate per case; error bars show SEM. *P<0.05, P<0.01, Wilcoxon rank sum test. Dotted lines indicate the threshold of sensitivity of detection. GAPDH, glyceraldehyde-3-phosphate dehydrogenase.

FIGS. 6A-6D depict the diversity and overlap of tissue and peripheral blood TCRs. (6A) The diversity of TCR in the blood greatly exceeds that in postvaccination tissue (n=3). (6B) TCRs that are shared between the tissue and blood of individual subjects who have been vaccinated comprise a greater fraction of tissue TCRs than peripheral blood TCRs. (6C) Heat maps depicting the frequency of shared TCRs in tissue and peripheral blood of three vaccinated (*) subjects (3007, 3009, and 3062) and one unvaccinated subject (0207CR), all of whom had HPV16⁺ CIN2/3. (6D) Heat maps depicting the frequency of shared TCRs in tissue and blood samples of two vaccinated subjects with shared human leukocyte antigen (HLA) alleles (3009 and 3062) and shared TCRs in tissue and blood samples of an unvaccinated (0207) and a vaccinated (3009) subject who shared HLA alleles.

FIGS. 7A-7B show intramuscular immunization with DNAE7 prime, followed by recombinant vaccinia (rVacE6E7) boost, induces HPV16 and E7-specific TH1 immune responses in the peripheral blood. (7A) Multiparametric flow cytometry was used to detect HPV16-and HPV18-specific regulation of interferon gamma (IFN-γ), CD107a, granzyme B (GrzB), and perforin (Prf). Representative gating strategy and response to HPV peptides are shown. Viable cells were isolated by forward vs. side scatter, then gated on CD3 expression. In all dot plots showing staining for surface and intracellular markers, negatively staining and positively staining populations are clearly identifiable. (7B) Co-expression of CD107a, granzyme B, and perforin in CD8+ T cell subset in response to HPV16 and HPV18 E6/E7. All subjects received two intramuscular DNA vaccinations at week 0 and week 4. Dose escalation of recombinant vaccinia (rVacE6E7) boost vaccination at week 8; subjects received either 1.6×10⁵ pfu (DDV1), 1.6×10⁶ pfu (DDV2), or 1.6×10⁷ pfu (DDV3). Subjects with complete histologic regression at week 15 are designated with a bar below the subject ID.

FIG. 8 depicts the intensity of lesional stromal CD8+ infiltrates increases significantly over a prospective 15-week window in vaccinated compared to unvaccinated subjects with HPV16+ CIN2/3 lesions. Quantitative image analysis of lesional stromal CD8+ infiltrates at study entry, and in lesion resections 15 weeks later, in an unvaccinated observational cohort (n=19) and in vaccinated subjects (n=12) (Wilcoxon rank sum test, p=0.0300).

FIGS. 9A-9D show the gating strategy and representative isotype controls for the flow cytometry studies of cervical T cells in FIG. 3. (9A) The gating strategy for surface and intracellular cytokine studies are shown. Viable cells were isolated by forward (FSC-H) vs. side (SSC-A) scatter. These cells were subsequently gated on CD3 expression. (9B) Representative isotype controls are shown. In general, gates were set using isotype controls so that less than 1% positive cells were present in each of the three positive staining quadrants. (9C) Gating strategy for FOXP3 intranuclear stains. Cells were gated first by scatter for viability and then a second gate based on negative staining for an isotype control was used to further exclude dead cells. Cells were then gated for CD3 expression.

### DETAILED DESCRIPTION OF THE INVENTION

The present inventive methods show qualitative and quantitative changes in the intensity, frequency, and localization of immune cells in CIN2/3 target lesions subsequent to systemic therapeutic vaccination. By conventional measures of vaccination “success,” specifically, vaccine antigen-specific T cell responses in the blood and/or complete regression of disease, these subjects would have been considered “failures.” However, in the target lesions, we measured marked changes in immune parameters known to be associated with better prognosis, despite modest detectable T cell responses to vaccine antigen in circulating peripheral blood T cells. Vaccination was followed by increased lesional intraepithelial CD8 infiltrates composed of clonally expanded TCRs disproportionately represented compared to in the blood and were associated with TLSs, evidence of proliferation and activation mediated by TCR engagement by cognate antigen, and expression of genes associated with a T_{H}1 response.

In accordance with an embodiment, the present invention provides a method for determining the effectiveness of an HPV vaccine regimen on a subject having a cervical intraepithelial neoplasia (CIN) comprising: a) obtaining at least one first tissue sample from the CIN of the subject; b) administering to the subject an effective amount of a first vaccine construct comprising HPV16 E7 antigen one or more times; c) administering to the subject an effective amount of a second vaccine construct comprising both E6 and E7 antigens from both HPV16 and HPV18; d) obtaining at least one or more second tissue samples from the CIN of the subject about 4 to about 24 weeks after step c); e)measuring the amount of CD8+ T cell infiltrates in the intraepithelial tissue of the first and second CIN samples; and f) determining that the HPV vaccine regimen is effective when the amount of CD8+ T cell infiltrates in the second tissue sample is increased when compared to the amount of CD8+ T cell infiltrates in the first tissue sample.

In some embodiments of the inventive methods, at step d), the at least one or more samples are obtained at 4 weeks to about 24 weeks after step c).

In some embodiments, at step f), when the amount of CD8+ T cell infiltrates in the second tissue sample is the same or decreased when compared to the amount of CD8+ T cell infiltrates in the first tissue sample, then the HOV vaccine regimen is determined to be ineffective.

In accordance with another embodiment, the inventive methods further comprise: step e1) measuring the quantity of Treg cells in the intraepithelial tissue of the first and second CIN samples; and f1) determining that the HPV vaccine regimen is effective when the ratio of the quantity of CD8+ T cells/Treg cells in the second tissue sample is increased when compared to the ratio of the quantity of CD8+ T cells/Treg in the first tissue sample.

In some embodiments, the methods further comprise at step f1), when the ratio of the quantity of CD8+ T cells/Treg cells in the second tissue sample is the same or decreased when compared to the ratio of the quantity of CD8+ T cells/Treg cells in the first tissue sample, then the HPV vaccine regimen is determined to be ineffective.

As used herein, the term “a first vaccine construct comprising HPV16 E7 antigen” means a DNA vaccine which expresses the HPV16 E6 antigen or functional epitope, such as “pNGVL4a-CRT/E7(detox) plasmid vaccine/HSP70” having DNA sequences which encode antigenic peptides, e.g., those derived from human papillomavirus (HPV), HPV-16 E7, as detailed in U.S. patent application Ser. No. 12/438,300, filed Jun. 7, 2010, and incorporated by reference herein.

As used herein, the term “a second vaccine construct comprising both E6 and E7 antigens from both HPV16 and HPV18” means a live virus expressing HPV16 and HPV18 E6 and E7 antigens, such as, for example “TA-HPV” as described in references (38-41). Boursnell M. E. G. et al. describes the construction and characterization of a recombinant vaccinia virus vector (TA-HPV) expressing the tumor antigens E6 and E7 from HPV16 or HPV18 in “Construction and characterization of a recombinant vaccinia virus expressing human papillomavirus proteins for immunotherapy of cervical cancer.” It was demonstrated that the recombinant virus, upon intraperitoneal administration to mice, has the capacity to prime a cytotoxic T lymphocyte (CTL) response against cells infected with the same virus vector or sensitized with a synthetic E7 peptide epitope.

The human papillomavirus is a DNA tumor virus that causes epithelial proliferation at cutaneous and mucosal surfaces. More than 100 different types of the virus exist, including approximately 30 to 40 strains that infect the human genital tract. Of these, there are oncogenic or high-risk types (16, 18, 31, 33, 35, 39, 45, 51, 52, and 58) that are associated with cervical, vulvar, vaginal, penile, oral, throat and anal cancers, and non-oncogenic or low-risk types (6, 11, 40, 42, 43, 44, and 54) that are associated with anogenital condyloma or genital warts. HPV 16 is the most oncogenic, accounting for almost half of all cervical cancers, and HPV 16 and 18 together account for approximately 70% of cervical cancers. HPV 6 and 11 are the most common strains associated with genital warts and are responsible for approximately 90% of these lesions.

In accordance with an embodiment, the HPV-associated disease treated by the inventive methods is cancer, including cervical, vulvar, vaginal, penile, oral, throat and anal cancers. In some embodiments, the cancer is cervical cancer.

In accordance with one or more embodiments, the pNGVL4a-CRT/E7(detox) vaccine and TA-HPV vaccines are given by injection, e.g., i.m., i.p., i.v., subcutaneously, gene gun, etc. In accordance with some embodiments, the inventive methods comprise vaccination intramuscularly.

In accordance with some embodiments, the inventive methods comprise administration of an adjuvant post-vaccination at the cutaneous and mucosal surfaces which are infected with HPV. In an embodiment, the mucosal tissue is the vaginal mucosa. In other embodiments, the cutaneous and mucosal surfaces are in the cervicovaginal tract.

In accordance with some embodiments, the tissue samples collected include dysplastic stromal and dysplastic epithelium in the lesions affected. In some embodiments of the inventive methods, increased intensity or amounts of CD8+ T-cell infiltrates in dysplastic cervical epithelium are correlative with positive clinical outcomes and lesional regression.

As used herein, the term “subject” can mean a subject suspected of having cervical cancer or suspected of having an increased risk of having a cervical neoplasia and can include a patient presenting cervical intraepithelial neoplasia (CIN), and/or low grade squamous intraepithelial lesion (LSIL) and/or high grade squamous intraepithelial lesion (HSIL), or any other abnormal Pap smear or cytological test.

As used herein, the term “subject” can also mean a subject suspected of having an HPV infection or HPV related disease, and also includes a subject that has either been exposed to HPV or has evidence of infection with HPV of any variant strain.

As used herein, the term “treat,” as well as words stemming therefrom, includes diagnostic and preventive as well as disorder remitative treatment.

As used herein, the term “subject” refers to any mammal, including, but not limited to, mammals of the order Rodentia, such as mice and hamsters, and mammals of the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human.

In accordance with another embodiment, the present invention provides a method for treating a subject having a cervical intraepithelial neoplasia (CIN) comprising: a) obtaining at least one first tissue sample from the CIN of the subject; b) administering to the subject an effective amount of a first vaccine construct comprising HPV16 E7 antigen one or more times; c) administering to the subject an effective amount of a second vaccine construct comprising both E6 and E7 antigens from both HPV16 and HPV18; d) obtaining at least one or more second tissue samples from the CIN of the subject about 4 to about 24 weeks after step c); e) measuring the amount of CD8+ T cell infiltrates in the intraepithelial tissue of the first and second CIN samples; and f) determining that the HPV treatment is effective when the amount of CD8+ T cell infiltrates in the second tissue sample is increased when compared to the amount of CD8+ T cell infiltrates in the first tissue sample.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention of cancer in a mammal Furthermore, the treatment or prevention provided by the inventive method can include treatment or prevention of one or more conditions or symptoms of the disease, e.g., cancer, being treated or prevented. Also, for purposes herein, “prevention” can encompass delaying the onset of the disease, or a symptom or condition thereof

In accordance with an embodiment, the present invention provides a method for determining the effectiveness of an HPV vaccine regimen with an adjuvant on a subject having a cervical intraepithelial neoplasia (CIN) comprising: a) obtaining at least one first tissue sample from the CIN of the subject; b) administering to the subject an effective amount of a first vaccine construct comprising HPV16 E7 antigen one or more times; c) administering to the subject an effective amount of a second vaccine construct comprising both E6 and E7 antigens from both HPV16 and HPV18, and an effective amount of an adjuvant; d) obtaining at least one or more second tissue samples from the CIN of the subject about 4 to about 24 weeks after step c); e) measuring the amount of CD8+ T cell infiltrates in the intraepithelial tissue of the first and second CIN samples; and f) determining that the HPV treatment is effective when the amount of CD8+ T cell infiltrates in the second tissue sample is increased when compared to the amount of CD8+ T cell infiltrates in the first tissue sample.

The term “adjuvant” as used herein, generally means a substance that increases the body's immune response to the vaccine. In accordance with the inventive methods, the adjuvants are applied directly to the site of the lesion, using topical or any other known means. In some embodiments, the adjuvants used in the methods of the present invention include toll-like receptor (TLR) ligands. TLR ligands are capable of generating stronger Trm recruitment. Currently, two TLR agonists are FDA approved for use in cancer patients in addition to imiquimod, the TLR4 agonist monophosphoryl lipid A (MPL), and the TLR2/4 agonist bacillus Calmette-Guérin (BCG). Also of interest is the TLR7/8 agonist, resiquimod, which is an imidazoquinoline like imiquimod, and has been shown to have antitumor effects. TLR9 agonists are being developed for clinical application in oncology and viral infections by Pfizer, Dynavax Technologies and GlaxoSmithKline among others. It will be understood by those of skill in the art that alternative adjuvants can be used in the inventive methods. In a specific embodiment, the adjuvant used is imiquimod (1-(2-methylpropyl)-1H-imidazo[4,5-c]quinolin-4-amine), also known under the trade names Aldara and Zyclara, and by Mochida as Beselna. It is also referred to as R-837.

In accordance with an embodiment, the present invention provides a method for treating a subject having a cervical intraepithelial neoplasia (CIN) comprising: a) obtaining at least one first tissue sample from the CIN of the subject; b) administering to the subject an effective amount of a first vaccine construct comprising HPV 16 E7 antigen one or more times; c) administering to the subject an effective amount of a second vaccine construct comprising both E6 and E7 antigens from both HPV16 and HPV18, and an effective amount of an adjuvant; d) obtaining at least one or more second tissue samples from the CIN of the subject about 4 to about 24 weeks after step c); e) measuring the amount of CD8+ T cell infiltrates in the intraepithelial tissue of the first and second CIN samples; and f) determining that the HPV treatment is effective when the amount of CD8+ T cell infiltrates in the second tissue sample is increased when compared to the amount of CD8+ T cell infiltrates in the first tissue sample.

In accordance with another embodiment, the present invention provides a treatment regimen for generating an immune response against human papillomavirus (HPV)-associated disease in a subject comprising administering to the subject a composition comprising a vaccine construct consisting of pNGVL4a-CRT/E7(detox) plasmid, followed by a Vaccinia based HPV16 and HPV18 E6 E7 vaccine booster, and subsequently administering to the subject an effective amount of a composition comprising an toll-like receptor 7 agonist to the site of the HPV-related disease or infection in the subject.

In accordance with a further embodiment, the present invention provides a method for quantifying the effectiveness of a treatment regimen for HPV induced cancer by measuring the accumulation of antigen-specific CD8+ T cells in the cervicovaginal tract of a subject infected with HPV in the cervicovaginal tract.

In some embodiments, the methods of the present invention can include pNGVL4a-CRT/E7(detox)/HSP70 and TA-HPV vaccines in conjunction and with a carrier. The carrier is preferably a pharmaceutically acceptable carrier. With respect to pharmaceutical compositions, the carrier can be any of those conventionally used and is limited only by chemico-physical considerations, such as solubility and lack of reactivity with the active compound(s), and by the route of administration. The pharmaceutically acceptable carriers described herein, for example, vehicles, adjuvants, excipients, and diluents, are well-known to those skilled in the art and are readily available to the public. It is preferred that the pharmaceutically acceptable carrier be one which is chemically inert to the active agent(s) and one which has no detrimental side effects or toxicity under the conditions of use.

Thus, in an embodiment, the present invention provides the use of a pharmaceutical compositions comprising a vaccine construct consisting of pNGVL4a-CRT/E7(detox)/HSP70 plasmid, TA-HPV, and an effective amount of a pharmaceutical composition comprising an adjuvant and a pharmaceutically acceptable carrier, as a medicament, preferably as a medicament for the treatment of an HPV-related disease or infection in a subject.

The choice of carrier will be determined in part by the chemical properties of vaccines as well as by the particular method used to administer the vaccines. Accordingly, there are a variety of suitable formulations of the pharmaceutical composition of the invention. The following formulations for parenteral, subcutaneous, intravenous, intramuscular, intraarterial, intrathecal, and intraperitoneal administration are exemplary and are in no way limiting. More than one route can be used to administer the vaccines, and in certain instances, a particular route can provide a more immediate and more effective response than another route.

Injectable formulations are in accordance with the present invention. The requirements for effective pharmaceutical carriers for injectable compositions are well-known to those of ordinary skill in the art (see, e.g., Pharmaceutics and Pharmacy Practice, J.B. Lippincott Company, Philadelphia, Pa., Banker and Chalmers, eds., pages 238-250(1982), and ASHP Handbook on Injectable Drugs, Trissel, 14th ed., (2007)).

For purposes of the invention, the amount or dose of pNGVL4a-CRT/E7(detox)/HSP70 vaccine, and the TA-HPV vaccine administered should be sufficient to effect, e.g., a therapeutic or prophylactic response, in the subject over a reasonable time frame. The dose will be determined by the efficacy of the vaccines and the condition of a human, as well as the body weight of a human to be treated.

Typically, the attending physician will decide the dosage of pNGVL4a-CRT/E7(detox) vaccine with which to treat each individual patient, taking into consideration a variety of factors, such as age, body weight, general health, diet, sex, to be administered, route of administration, and the severity of the condition being treated. By way of example and not intending to limit the invention, the dose of pNGVL4a-CRT/E7(detox)/HSP70 vaccine can be about 100 μg to about 10 mg of pNGVL4a-CRT/E7(detox) vaccine to the subject being treated. In some embodiments, the dosage range is about 2-5 mg of pNGVL4a-CRT/E7(detox) vaccine. By way of example and not intending to limit the invention, the dose of TA-HPV Vaccinia vaccine can be between 1×10⁵ PFU to 5×10⁷ PFU.

The attending physician will also decide the dosage of amount of adjuvant with which to treat each individual patient, taking into consideration a variety of factors, such as age, body weight, general health, diet, sex, to be administered, route of administration, and the severity of the condition being treated. In some embodiments the dosage range is about 100 μg to about 1000 μg of imiquimod. In an embodiment, the dosage range of imiquimod is about 200 μg to about 400 μg. The formulations can vary with the route of administration.

In certain embodiments, the adjuvant is administered topically, to the site of the HPV-related disease in the subject. In a specific embodiment, the adjuvant is imiquimod, which is administered topically in a cream formulation.

It is contemplated that the inventive methods can be used for assessing or determining the effect of HPV immunotherapeutics in conjunction with additional biological agents.

An “active agent” and a “biologically active agent” are used interchangeably herein to refer to a chemical or biological compound that induces a desired pharmacological and/or physiological effect, wherein the effect may be prophylactic or therapeutic. The terms also encompass pharmaceutically acceptable, pharmacologically active derivatives of those active agents specifically mentioned herein, including, but not limited to, salts, esters, amides, prodrugs, active metabolites, analogs and the like. When the terms “active agent,” “pharmacologically active agent” and “drug” are used, then, it is to be understood that the invention includes the active agent per se as well as pharmaceutically acceptable, pharmacologically active salts, esters, amides, prodrugs, metabolites, analogs etc. The active agent can be a biological entity, such as a virus or cell, whether naturally occurring or manipulated, such as transformed.

The biologically active agent may vary widely with the intended purpose for the composition. The term active is art-recognized and refers to any moiety that is a biologically, physiologically, or pharmacologically active substance that acts locally or systemically in a subject. Examples of biologically active agents, that may be referred to as “drugs”, are described in well-known literature references such as the Merck Index, the Physicians' Desk Reference, and The Pharmacological Basis of Therapeutics, and they include, without limitation, medicaments; vitamins; mineral supplements; substances used for the treatment, prevention, diagnosis, cure or mitigation of a disease or illness; substances which affect the structure or function of the body; or pro-drugs, which become biologically active or more active after they have been placed in a physiological environment.

Specific examples of useful biologically active agents the above categories include: anti-neoplastics such as androgen inhibitors, alkylating agents, nitrogen mustard alkylating agents, nitrosourea alkylating agents, antimetabolites, purine analog antimetabolites, pyrimidine analog antimetabolites, hormonal antineoplastics, natural antineoplastics, antibiotic natural antineoplastics, carboplatin and cisplatin; nitrosourea alkylating antineoplastic agents, such as carmustine (BCNU); antimetabolite antineoplastic agents, such as methotrexate; pyrimidine analog antineoplastic agents, such as fluorouracil (5-FU) and gemcitabine; hormonal antineoplastics, such as goserelin, leuprolide, and tamoxifen; natural antineoplastics, such as aldesleukin, interleukin-2, docetaxel, etoposide, interferon; paclitaxel, other taxane derivatives, and tretinoin (ATRA); antibiotic natural antineoplastics, such as bleomycin, dactinomycin, daunorubicin, doxorubicin, and mitomycin; vinca alkaloid natural antineoplastics, such as vinblastine and vincristine.

Other biologically active agents can include peptides, proteins, and other large molecules, such as interleukins 1 through 18 including mutants and analogues; interferons α, γ, and which may be useful for cartilage regeneration, hormone releasing hormone (LHRH) and analogues, gonadotropin releasing hormone transforming growth factor (TGF); fibroblast growth factor (FGF); tumor necrosis factor-α); nerve growth factor (NGF); growth hormone releasing factor (GHRF), epidermal growth factor (EGF), connective tissue activated osteogenic factors, fibroblast growth factor homologous factor (FGFHF); hepatocyte growth factor (HGF); insulin growth factor (IGF); invasion inhibiting factor-2 (IIF-2); bone morphogenetic proteins 1-7 (BMP 1-7); somatostatin; thymosin-α-γ-globulin; superoxide dismutase (SOD); and complement factors, and biologically active analogs, fragments, and derivatives of such factors, for example, growth factors.

In unvaccinated patients with solid tumors, the quality of immune response in the tumor microenvironment has been shown to provide prognostic information that is not readily obvious in the blood (16, 26). Relevant parameters associated with improved clinical outcomes in treatment settings using conventional anticancer modalities include intratumoral CD3⁺CD8⁺ infiltrates, T cells with an antigen-experienced phenotype (CD45RO⁺), a T_{H}1 molecular signature (IFNγ, IL-12, Tbet, and IRF1), cytotoxic effector molecules (granzymes, perforin, and granulysin), chemokines that play a role in recruiting effector immune cells (CX3CL1, CXCL9, CXCL10, CCL5, and CCL2), and TLSs (16, 26-28). Colocalized high endothelial venules, a vessel characteristic of lymph node structures, are also detected in tumor stroma in regions with T cell infiltrates and are also independently associated with improved overall survival (16, 29). Efforts to standardize a tissue immune “phenotype” are under way (30). The marked changes in cervical mucosal tissue in the subjects using the inventive methods provided herein show that many features associated with better prognosis had been induced by vaccination. These included a constellation of histologic changes in conjunction with molecular analyses that together suggest ongoing adaptive responses that are not only localized in tissue compared to the blood but also qualitatively and quantitatively different from unvaccinated CIN2/3 lesions.

The presence of an immune response in the tissue that is not readily detectable in the peripheral blood is likely to reflect both characteristics of the immune response to HPV E6 and E7 antigens, as well as issues associated with therapeutic vaccination. Although E6 and E7 are foreign proteins, not “self,” to date, they have not appeared to be highly immunogenic, at least in the context of most previously tested vaccine vectors. Whether this lack of “immunogenicity” reflects the underlying T cell repertoire or limitations of the processing and presentation of epitopes derived from these proteins remains to be determined The problem of immunogenicity may be addressed in part by emerging clinical data from trials testing therapeutic DNA vaccination delivered via electroporation (31). Recent data reporting safety, tolerability, and immunogenicity of DNA vaccination targeting HPV16 and HPV18 E6 and E7, delivered using electroporation in subjects without evidence of disease, demonstrate significant induction of antigen-specific cytotoxic T cells, detectable directly ex vivo. Phase 2 studies of vaccination before resection, similar to the present inventive methods in this study, are ongoing (NCT01304524).

As used herein, the term “generating an immune response against human papillomavirus (HPV)-associated disease” means the accumulation of antigen-specific CD8+ T cells in the site of infection or disease. The term can also include the migration of CXCR3+ CD8+ T cells to the site of infection or disease.

The present findings suggest that there is much to be learned about the dynamics of responses to vaccination in humans, particularly in settings in which there is a tissue reservoir of the antigen being targeted. Use of the inventive methods provided herein resulted in a finding that the intensity or amount of tissue CD8⁺ infiltrates before vaccination was associated with the magnitude of peripheral blood responses 8 and 12 weeks after the boost vaccination was unexpected, and show that the vaccine responses detectable in the blood could represent a boosted anamnestic response, rather than a de novo primary response to vaccination. The results also suggest that the timing of when the peripheral blood is drawn after vaccination affects the likelihood of detecting antigen-specific T cells, because the response appears to sequester significantly in the site of the persistent antigen.

Although the ability to compare pre- and post-vaccination tissue specimens in vaccinated subjects did provide a valuable opportunity to query tissue sections for proof of principle for the inventive methods, resection in this time frame essentially censored histologic endpoint analyses. The clinical endpoints were dictated by a clear priority for patient safety. Therapeutic resections were scheduled for 7 weeks after the third vaccination because this time frame is within the standard of care for treating CIN2/3 in a non-investigational setting. However, these findings have practical implications for the design and interpretation of clinical trials testing therapies for pre-invasive HPV disease. Because intraepithelial infiltrates are associated with subsequent regression, and because an increase in intraepithelial CD8+ infiltrates was associated with detectable responses to HPV in the blood, the present invention of providing a monitoring strategy that incorporated features of a “tissue response,” such as increased intraepithelial CD8 infiltrates, in conjunction with a measure of “peripheral blood response,” such as increased ELISpot response to vaccine antigen, can be used to inform clinical studies of therapeutic HPV vaccines. The quantitative image analyses described herein can be performed using reagents and methods available in any surgical pathology laboratory.

Tissue-based analyses of clinical specimens have been limited by the small absolute size of available tissue, because the first principle is to not compromise the ability to make an accurate pathological diagnosis. The development of sophisticated, high-throughput analytic technologies, as used in the inventive methods, now provides opportunities to derive a great deal of information directly ex vivo from limited specimens of unmanipulated cells. The present invention holds great promise for enhancing our understanding of mechanisms of immune cell recruitment, repertoire, functional polarization, and cell retention, but must be informed and guided meticulously by tissue image analyses to minimize the odds of deriving data sets that could obscure or mask a dynamic clinical picture. In the case of our specimens, assessment of the repertoire of tissue-localized T cells was technically challenging because the absolute number of cells that could be isolated from small explants was too low to carry out conventional measures of specificity, such as ELISpot or flow cytometric analyses of cells stimulated with antigen. Although ex vivo expansion of tissue T cells would have provided more cells, published methods indicate that the cells that expanded would have represented a subset of cervical tissue T cells, in terms of repertoire, as well as phenotype and function (32, 33). The paired tissue and blood TCR sequence analyses used in the present inventive methods were carried out using genomic DNA extracted from unmanipulated cells. Data derived from carefully selected regions of tissue subsets will yield different information than those derived from whole tissue samples. These types of studies, although time-consuming and laborious, can be used in the inventive methods, and can be useful to expanding our understanding of mechanisms by which peripheral tissues may sustain relevant immune responses to peripheral manipulation, for example, vaccination, including epitope spreading (17, 34, 35). Use of the inventive methods may be especially important in the case of tumors with high rates of mutation, such as adenocarcinomas and squamous cancers of the lung (36). Tissue-based studies using the inventive methods can also identify therapeutic barriers in the lesional microenvironment that can be targeted in concert with immune-based therapies for HPV disease.

Intraepithelial lesions associated with HPV are clinically indolent and are also directly accessible—two features that could also allow for making clinical management decisions based on tissue monitoring. Because colposcopically directed biopsies are much less invasive or destructive than a cone resection, patients whose lesional biopsies showed evidence of an effector immune response could potentially be monitored and saved from more invasive surgery, whereas patients whose lesions did not manifest an effector response could proceed to therapeutic resection. However, these kinds of treatment management decisions will be predicated on a better understanding of what constitutes a clinically meaningful tissue immune response.

### EXAMPLES

The clinical trial protocols were reviewed and approved by the Institutional Review Board at Johns Hopkins Hospital (NA_{—}00002176, NA_{—}00023308). All study participants gave written informed consent before undergoing screening for study eligibility and enrollment. This trial is listed at clinicaltrials.gov (NCT00788164).

Patient demographics and trial design.

Twelve subjects with HPV16+ CIN2/3 were enrolled in this phase 1, open-label clinical trial designed to assess the safety, tolerability, and immunogenicity of priming vaccination with DNA vaccine targeting HPV16 E7, followed by escalating doses of a boost vaccination with recombinant vaccinia targeting HPV 16 and HPV18E6 and E7 (TA-HPV). Standard therapeutic resection of lesions was performed 7 weeks after the boost vaccination. Eligibility criteria included ability to give informed consent, immune competence, HIV seronegativity, nonpregnant status, and presence of residual, visible lesion after a colposcopically directed, biopsy-confirmed diagnosis of CIN2/3. Patients included in this analysis had lesions associated with HPV16, the genotype most commonly associated with cervical cancer and its precursor lesions. Eligible subjects were assigned to a treatment cohort and received intramuscular vaccination at study weeks 0 and 4 with 3mg of pNGVL4a-sig/E7(detox)/HSP70. At week 8, patients received one of three rVacE6E7 (TA-HPV) doses: 1.6×10⁵ PFU (cohort DDV1; n=3), 1.6×10⁶ PFU (cohortDDV2; n=3), or 1.6×10⁷ PFU (cohort DDV3; n=6), administered intramuscularly in the deltoid muscle. After vaccination with TA-HPV, the vaccination site was covered with an occlusive dressing (Opsite Flexigrid, Smith & Nephew).

Patients were observed for 30 minutes after each vaccination. Patients were given diary cards and instructed to record any local or systemic AEs daily for 4 weeks. All safety data, including injection site reactions and AEs, were reviewed by the institutional Data Safety and Monitoring Board and submitted for regular review to the U.S. Food and Drug Administration. Standard therapeutic resection of the cervical squamocolumnar junction was performed at week 15.

Primary endpoints included standard safety and tolerability endpoints as defined in CTCAE v4.0. Secondary endpoints included histologic regression, defined as no CIN2/3 in the resection specimen, and immune response to vaccine antigens Immunogenicity was assessed by HPV16 and HPV18 E6/E7-specific IFN-g ELISpot assay and flow cytometric assays for IFN-g, granzyme B, and perforin with cryopreserved PBMCs obtained at screening (TO), at study weeks 8 to 10 (T2), at study week 15 (T3), and at study week 19 (T4).

Study vaccines.

pNGVL4a-sig/E7(detox)/HSP70 is a plasmid DNA construct used previously as a stand-alone vaccination in a previous phase 1 clinical trial in a similar cohort (37). It was manufactured under Good Manufacturing Practices by the National Cancer Institute (NCI) Rapid Access to Interventional Development program and met all acceptance criteria for release. It is composed of a closed circular DNA plasmid expressing HPV16 E7 mutated at amino acids 24 and 26, linked to sequences coding for sig and for HSP70.

TA-HPV is a live attenuated recombinant Vaccinia virus expressing HPV16 and HPV18 E6 and E7 (38). It is composed of fused E6 and E7 open reading frames of HPV 16 and HPV18, each under the control of a vaccinia promoter, in the Wyeth strain of vaccinia. The E7 sequence is mutated so that capacity to bind to retinoblastoma protein is abrogated.

The fused gene products have been shown to have no transforming activity. Vaccine was manufactured and released by Cantab (now Celtic Pharma). Production, analysis, and storage were carried out under Good Manufacturing Practice standards. Clinical trials testing this vaccine in a range of patient cohorts have been previously reported (38-71). Vaccination in estimated doses of 1.6×10^{5,} 1.6×10⁶, or 1.6×10⁷ PFU was administered intramuscularly in the deltoid muscle.

Endpoint evaluations.

Colposcopy was performed at week 15, 7 weeks after the third vaccination. A standard therapeutic resection of the cervical squamocolumnar junction (either a cold knife conization or a loop electrosurgical excisional procedure) was performed. A frozen section was obtained from one radial section of the resection, and only if the pathologist did not have any suspicion of invasion, the flash-frozen tissue was banked, and an adjacent 2-mm sliver was reserved for explant cultures as previously described (11).

Immunologic analyses of PBMC samples.

PBMCs were prioritized first to the IFN-γ ELISpot assay. Sufficient quantities were available to conduct assays using peptides spanning the four antigens separately, to allow estimation of individual response against each antigen. In one subject in the full-dose boost cohort, two time points were not available, because the patient was unable to keep those study visits. The remaining samples were used for ICS assays. No subject was excluded for reasons other than sample availability.

ELISpot assays were performed by the University of Pennsylvania Human Immunology Core Facility using a qualified protocol as previously described (42). The standard ELISpot protocol with 24-hour peptide stimulation was previously cross-validated across different laboratories (43) and was adapted for use with HPV-specific peptide pools. Differences between the above-referenced protocol and the protocol used in the current study relate only to the use of HPV peptides as the stimulating antigen. Specifically, the current protocol used two sets of peptides, each containing 15-amino acid residues overlapping by 8 amino acids representing the entire consensus E6/E7 fusion protein sequence of HPV16 or HPV18, and were pooled at a concentration of 2 mg/ml per peptide into two pools, spanning the length of the E6 and E7 antigens, respectively (44, 45). The average number of SFUs counted in R10 wells was subtracted from the average in individual HPV peptide wells and then adjusted to 1×10⁶ PBMCs for each HPV peptide pool. To summarize the T cell ELISpot data, immune responses to each individual antigen were reported. For each time point, the mean number of SFUs from triplicate wells with PBMCs incubated with medium alone (background) was subtracted from the means of PBMCs stimulated with HPV16 or HPV18 E6 or E7 peptides. After subtracting medium control, a positive response was defined as at least 20 SFUs/10⁶ PBMCs and greater than two times the SD of the pre-vaccination antigen specific response.

Intracellular cytokine staining.

ICS was performed as previously described (46) using the following markers: CD107a-PECy7, CD14-Pacific Blue, CD16-Pacific Blue, CD8-APC (allophycocyanin), CD4-PerCPCy5.5, IFN-g-FITC (fluorescein isothiocyanate), and CD45RO-AF700 (BDBiosciences); CD19-Pacific Blue and granzyme B-PE (phycoerythrin) Texas Red (Invitrogen); CD27-PECy5 (eBioscience); and perforin-PE (Abeam). Prepared cells were acquired with an LSR II flow cytometer equipped with BD FACSDiva software (BD Biosciences). Acquired data were analyzed with FlowJo software version 7.6.3 (Tree Star). All samples used for this assay were from a pre-immunization time point (all subjects tested) and from the week 8 to 10 time point, the week 15 time point, and the week 19 time point. Staining was performed once per time point per sample listed.

IHC staining of tissue sections.

Tissue in paraffin blocks that was residual after histopathologic diagnoses had been finalized was prioritized first for IHC evaluation. After sections were obtained for CD8, Foxp3, and Ki67 IHC, sections were cut for laser capture microdissection from tissue blocks with sufficient residual material. Five-micrometer sections were cut from formalin-fixed, paraffin-embedded tissue from prevaccination (diagnostic biopsy) and from the postvaccination resection (7 weeks after the third vaccination). Heat-based Ag retrieval was performed for 30 minutes, followed by blocking endogenous peroxidase with 0.3% H₂O₂ and incubation with primary antibody. Primary antibodies were detected with the PowerVision+Poly-HRPIHCDetection System (LeicaBiosystems), as per the manufacturer's instructions. After incubation with detection reagents, diaminobenzidine was used as a chromogen, and Harris hematoxylinwas used as a counterstain. The following primary antibodies were used:CD8(clone 4B11, Leica Biosystems, dilution 1:500, incubation overnight at 4° C.), Tbet (clone 4B10, BDBiosciences, dilution 1:500, incubation overnight at 4° C.), Foxp3 (clone 236A/E7, eBioscience, dilution 1:50, incubation overnight at 4 ° C.), Ki67 (clone MIB-1, Dako, dilution 1:400, incubation overnight at 4° C.), PNAd (clone MECA-79, BD Biosciences, dilution 1:100, incubation overnight at 4° C.), CD3 (clone SP7, Novus Biologicals, dilution 1:100, incubation for 4 hours at room temperature), and CD20 (clone L26, Dako, dilution 1:600, incubation for 30 minutes at room temperature).

Quantitation of intensity and colocalization of immune cell subsets in tissue sections of the target tissue.

Images were captured with a Nikon E600 microscope/Plan Fluor ×20/0.50 ocular and a DXM1200f Nikon digital camera. ROIs were delineated in normal epithelium, stroma immediately beneath normal epithelium, CIN2/3 epithelium, and stroma immediately beneath CIN2/3 with the NIS Elements AR3 imaging software (Nikon). Chromogen was quantitated by normalization against the area of the ROI (mm²) At least 3 and up to 10 discrete ROIs were quantified in each compartment in each case. The mean density of chromogen staining for each compartment for each case was calculated and used for statistical comparisons between groups.

Phenotype analyses of tissue T cells derived from fresh tissue explants.

Primary tissue explants were obtained from surgical resection specimens. Normal mucosa obtained on banking protocol governing acquisition of residual tissue after standard diagnostic sections had been obtained. Tissue explants were obtained from standard therapeutic resections of the cervical squamocolumnar junction, and only if the surgical pathologists had no suspicion of invasive disease on a frozen section, a 2-mm section of fresh tissue immediately adjacent to the frozen section was reserved. Primary tissue explants were cultured using the method of Clark et al. (15). Flow cytometry analysis of cervical T cells was performed using directly conjugated monoclonal antibodies obtained from BioLegend (CD3, CD4, CD8, CD45RO, CD45RA, and L-selectin/CD62L), R&D Systems (CCR7), and the National Institutes of Health (NIH) AIDS Reagent Program (a4b7 and ACT-1). Analysis of flow cytometry samples was performed on BD Biosciences FACScan or FACSCanto instruments, and data were analyzed with FACSDiva software (version 5.1, BD Biosciences).

Laser capture microdissection.

Laser capture microdissection was used to segregate epithelium and subjacent stroma from 10-mm sections cut from paraffin-embedded tissue. Sections were mounted onto PALMMembraneSlides that had been pretreated with RNase-ZAP and irradiated (Zeiss MembraneSlide 1.0 PEN; 415101-4401-050). Laser capture microdissection was performed with the Axiovert 200 M (PALM MicroBeam) microdissection system (Zeiss), using PALM Robo v3.2 software. ROIs were delineated by free-hand tracing and catapulted into the cap of a 0.5-ml AdhesiveCap 500 opaque tube (Zeiss 415101-4400-255).

Quantitative reverse transcription polymerase chain reaction.

RNA samples were prepared from microdissected formalin-fixed sections with the AllPrepDNA/RNAFFPE kit (Qiagen). The amount and purity of RNA were verified with the NanoDrop ND-1000 spectrophotometer (Isogen) and calculated by the ratio of the readings at 260 and 280 nm (A260/280). The average A260/A280 ratio for the samples tested was 1.92 (range, 1.6 to 2.2), and the average concentration of RNA was 10.1 ng/ml (range, 4.2 to 14.6 ng/ml). First-strand complementary DNA (cDNA) synthesis was produced with the ABI High Capacity RNA-to-cDNA Kit (Applied Biosystems). Pre amplification was carried out with 300 ng of cDNA, TaqMan Custom PreAmp pools, and TaqMan PreAmpMaster Mix (Applied Biosystems). The reaction conditions were as follows: denaturation step at 95° C. for 10 minutes, followed by 14 cycles of 95° C. for 15 seconds and 60° C. for 4 minutes. Upon completion, samples were immediately removed from the thermal cycler, placed on ice, and diluted 1:20. Quantitative reverse transcription polymerase chain reaction (qRT-PCR) was carried out on pre amplified cDNA samples in a final volume of 20 ml in 96-well plates with the Applied Biosystems Prism 7900HT TaqMan instrument. The reaction mixture was composed of diluted preamplified cDNA sample, TaqMan Gene Expression Master Mix, and TaqMan Gene Expression Assays for CD4 (Hs00181217_m1), CD8B (Hs00174762_m1), Foxp3 (Hs01085834_m1), TBX21 (Hs00203436_m1), IFNG (Hs00989291_m1), IFNB1 (Hs01077958_s1), CCR7 (Hs01013469_m1), PRF1 (Hs00169473_m1), GZMB (Hs00188051_m1), CXCR3 (Hs01847760_s1), CD25 (Hs00907777_m1), CD69 (Hs00934033_m1), CD137 (Hs00155512_m1), GATA3 (Hs00231122_m1), RORC (Hs01076112_m1), and BCL6 (Hs00277037_ml) (Applied Biosystems). Cycling conditions were as follows: denaturation at 95° C. for 10 minutes, followed by 40 cycles of 95° C. for 15 seconds and 60° C. for 60 seconds. GAPDH was used as an internal control gene to normalize the reaction for the amount of RNA added to the reverse transcription reactions. Each real-time PCR was performed in duplicate. The entire pre amplification/qRT-PCR procedure was repeated on a subset of genes for which sufficient residual material permitted, with similar results.

TCR sequencing.

The sequences for both the TCRB CDR3 regions were delineated according to the definition established by the International ImMunoGeneTics collaboration (47). Sequences that did not match CDR3 sequences were removed from the analysis. A standard algorithm was used to identify which V, D, and J segments contributed to each TCRB CDR3 sequence (47). In both the blood and the tissue samples, the sample of T cells was a subsampling of a larger set of T cells. The total T cell repertoire was estimated using the unseen species model, a computational approach originally developed by Fisher et al. (48), and adapted using TCRB CDR3 sequences as individual species (21).

Statistical analyses.

The sample size for the clinical protocol was based on a standard 3 + 3 dose-finding design with three dosing levels and an additional three individuals at the maximally tolerated dose. Given the low toxicity levels observed in previous DNA vaccine studies, the expected sample size was 12 patients: 9 for dose escalation and 3 for the maximally tolerated dose. Within-subject quantitative tissue analyses of immune cell subsets were assessed using Wilcoxon signed rank tests. Differences in quantitative endpoints between groups were assessed using the Wilcoxon rank sum test. Correlations were determined using Pearson correlations. Two-sided testing with a significance level of P<0.05 was used for all analyses.

### Example 1

Heterologous prime-boost vaccination targeting HPV16 E6/E7 is safe and tolerable in subjects with HPV16+ CIN2/3 lesions.

Healthy subjects with HPV 16-associated CIN2/3 underwent peripheral vaccination with a heterologous DNA prime-recombinant vaccinia vector-based boost vaccination regimen administered intramuscularly in the deltoid muscle before a standard therapeutic resection. The regimen included two priming vaccinations with a DNA vaccine expressing HPV16 E7 (DNAE7) at study weeks 0 and 4, followed by a recombinant vaccinia boost expressing HPV16 and HPV18 E6 and E7 (rVacE6E7; TA-HPV) at study week 8. At week 15, 7 weeks after the boost vaccination, subjects underwent a standard therapeutic resection of the cervical squamocolumnar junction. A total of 12 patients in three treatment arms were evaluated (Table 1).

Demographics are presented in Table 2. All subjects received 3 mg of DNAE7 at entry and at week 4. At week 8, patients received one of three rVacE6E7 doses: 1.6×10⁵ plaque-forming units (PFU) (cohort DDV1; n=3), 1.6×10⁶ PFU (cohort DDV2; n=3), or 1.6×10⁷ PFU (cohort DDV3; n=6). Peripheral blood mononuclear cells (PBMCs) were obtained at baseline and at weeks 8, 15, and 19. Rates and severity of injection site reactions during the 30 days after each vaccination and frequency of adverse events (AEs) were recorded (data not shown). All reported AEs were mild, and injection site reactions resolved without sequelae or intervention. One of three patients in each of the DDV1 and DDV2 cohorts had complete histologic regression at time of resection. In the highest dose cohort (DDV3), three of six patients had a complete histologic response.

TABLE 1
Treatment cohorts. CR, complete regression.

| Treatment group | DNAE₇ (mg) | Vac_{E6E7} (PFU) | n | CR |
|---|---|---|---|---|
| DDV1 | 3 | 1.6 × 10⁵ | 3 | 1/3 (33%) |
| DDV2 | 3 | 1.6 × 10⁶ | 3 | 1/3 (33%) |
| DDV3 | 3 | 1.6 × 10⁷ | 6 | 3/6 (50%) |

TABLE 2
Demographics of study participants. B, black; W, white; CR, complete
regression; NR, not complete regression.

| Patient | | Age | | | | | | | | | DRB1 | | | | | DQB1 | |
|---|---|---|---|---|---|---|---|---|---|---|---|---|---|---|---|---|---|
| ID | HPV | (years) | Race | | A locus | | B locus | | C locus | | locus | | DRB3 | DRB4 | DRB5 | locus | |
| 1.1 | 16 | 26 | W | CR | A* | A* | B* | B* | C* | C* | DRB1* | | | DRB4* | | DQB1* | |
| | | | | | 02:01 | 24:02 | 13:02 | 44:02 | 05:01 | 06:02 | 07:01 | | | 01:xx | | 02:02 | |
| 1.2 | 16 | 24 | W | NR | A* | A* | B* | B* | C* | C* | DRB1* | DRB1* | | DRB4* | | DQB1* | DQB1* |
| | | | | | 02:01 | 29:02 | 38:01 | 44:03 | 12:03 | 16:01 | 04:04 | 07:01 | | 01:xx | | 02:02 | 03:02 |
| 1.3 | 16, | 22 | W | NR | A* | A* | B* | B* | C* | C* | DRB1* | DRB1* | | DRB4* | DRB5* | DQB1* | DQB1* |
| | 6, | | | | 03:01 | 31:01 | 07:02 | 40:01 | 03:04 | 07:02 | 04:04 | 15:01 | | 01:xx | 01:xx | 03:02 | 06:02 |
| | 52, | | | | | | | | | | | | | | | | |
| | 55 | | | | | | | | | | | | | | | | |
| 2.1 | 16 | 25 | B | CR | A* | A* | B* | B* | C* | C* | DRB1* | DRB1* | | DRB4* | | DQB1* | DQB1* |
| | | | | | 29:02 | 30:01 | 15:03 | 42:01 | 02:10 | 17:01 | 04:05 | 10:01 | | 01:xx | | 03:02 | 05:01 |
| 2.2 | 16, | 26 | W | NR | A* | | B* | B* | C* | C* | DRB1* | DRB1* | | | | DQB1* | |
| | 62, | | | | 25:01 | | 35:01 | 44:02 | 04:01 | 05:01 | 01:01 | 10:01 | | | | 05:01 | |
| | 84 | | | | | | | | | | | | | | | | |
| 2.3 | 16 | 26 | W | NR | A* | | B* | B* | C* | C* | DRB1* | DRB1* | 3* | 4* | | DQB1* | |
| | | | | | 11:01 | | 07:02 | 51:01 | 07:02 | 15:02 | 04:01 | 11:01 | 02:xx | 01:xx | | 03:01 | |
| 3.1 | 16 | 44 | W | NR | A* | A* | B* | B* | C* | C* | DRB1* | | | | DRB5* | DQB1* | DQB1* |
| | | | | | 03:01 | 29:02 | 07:02 | 27:02 | 05:01 | 07:02 | 15:01 | | | | 01:xx | 05:03 | 06:02 |
| 3.2 | 16, | 24 | W | NR | A* | A* | B* | B* | C* | C* | DRB1* | DRB1* | | DRB4* | | DQB1* | DQB1* |
| | 59, | | | | 02:01 | 03:01 | 40:01 | 44:03 | 03:04 | 16:01 | 04:04 | 07:01 | | 1:xx | | 02:02 | 03:02 |
| | 66 | | | | | | | | | | | | | | | | |
| 3.3 | 16 | 27 | W | CR | A* | A* | B* | B* | C* | C* | DRB1* | DRB1* | 3* | | 5* | DQB1* | DQB1* |
| | | | | | 02:01 | 03:01 | 07:02 | 40:02 | 02:02 | 07:02 | 11:01 | 15:01 | 02:xx | | 01:xx | 03:01 | 06:02 |
| 3.4 | 16, | 30 | W | CR | A* | A* | B* | B* | C* | C* | DRB1* | DRB1* | | 4* | | DQB1* | DQB1* |
| | 18 | | | | 24:02 | 32:01 | 15:01 | 40:02 | 03:03 | 15:02 | 04:01 | 04:04 | | 01:xx | | 03:01 | 03:02 |
| 3.5 | 16 | 27 | W | NR | A* | A* | B* | B* | C* | C* | DRB1* | DRB1* | 3* | | 5* | DQB1* | DQB1* |
| | | | | | 02:66 | 24:02 | 07:02 | 51:01 | 07:02 | 15:02 | 14:01 | 15:01 | 02:xx | | 01:xx | 05:03 | 06:02 |
| 3.6 | 16, | 49 | W | CR | A* | | B* | | C* | | DRB1* | | 3* | | | DQB1* | |
| | 62, | | | | 01:01 | | 08:01 | | 07:01 | | 03:01 | | 01:xx | | | 02:01 | |
| | 84 | | | | | | | | | | | | | | | | |

### Example 2

Therapeutic vaccination elicits HPV-specific cellular immune responses in the blood.

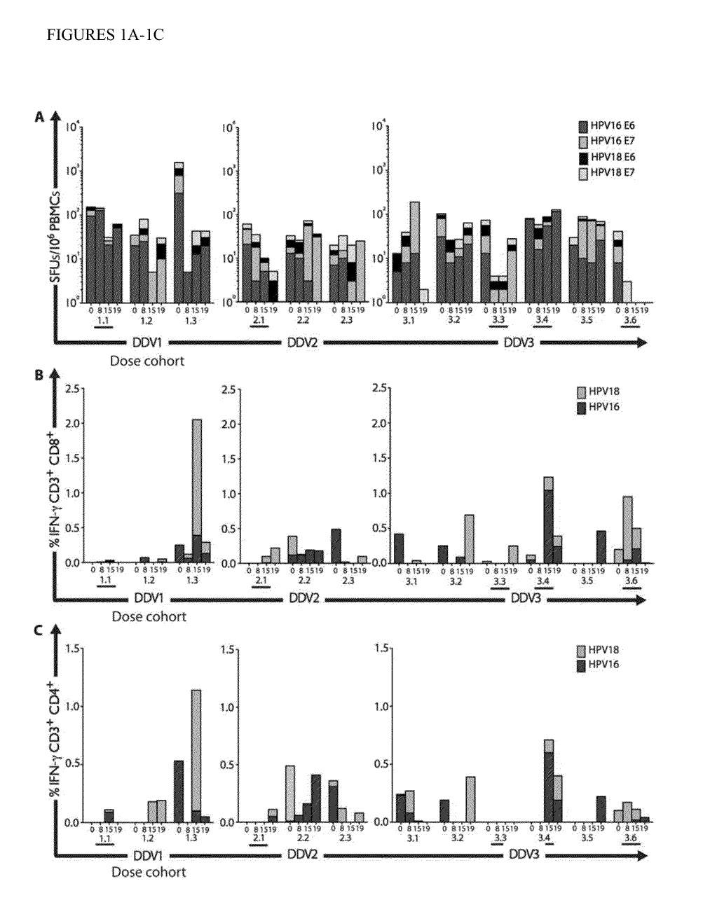

Standard interferon-γ (IFN-γ) enzyme-linked immunospot (ELISpot) assays (12, 13) were performed to determine the number of antigen-specific IFN-γ-secreting cells in response to stimulation with HPV16 or HPV18 E6 and E7 peptide pools. As shown in FIG. 1, after three vaccinations, one of three subjects who received the low-dose boost vaccination, all three subjects who received the intermediate-dose boost vaccination, and three of six subjects who received the full-dose boost vaccination mounted vaccine-induced HPV16-specific cellular immune responses. Overall, seven subjects (58%) developed cellular immune responses detectable by ELISpot. Peak responses were modest, in the range of 50 to 150 spot-forming units (SFUs) per 10⁶ PBMCs. However, using this assay, increased responses were restricted to HPV16 E7, suggesting that the DNA vaccine, which targeted only the HPV 16 E7 antigen, might indeed have a priming effect.

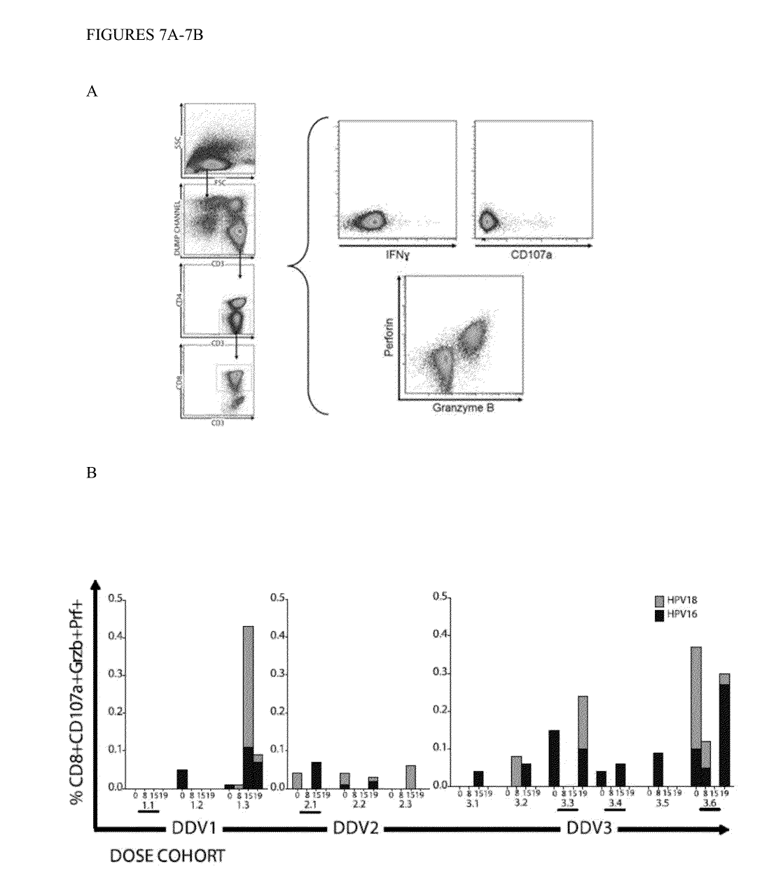

Intracellular cytokine staining (ICS) assays identified HPV16- and/or HPV18-specific IFN-γ-producing CD8 and CD4 T cells in one of three subjects in the low-dose boost cohort, in two of three subjects in the intermediate-dose boost cohort, and in four of six subjects in the full-dose boost cohort (FIG. 1). Sufficient cells were available to perform additional phenotyping studies on T cells cultured briefly with either HPV 16 or HPV 18 peptides, which demonstrated induction of T helper 1 (T_{H}1)-biased responses in CD3⁺CD4⁺ and CD3⁺CD8⁺ cells (FIGS. 1B and 1C, and FIG. 7). One subject (3009) in the low-dose cohort had a preexisting endogenous immune response to HPV18. The kinetics of response in this patient and in subjects in the full-dose cohort suggest that the timing of the peripheral blood draws in relationship to vaccination is an important variable in detecting responses. Most of the vaccine responses were identified in the week 15 specimens (that is, after the boost vaccination), as opposed to the earlier time point (weeks 8 to 10), which would have reflected responses after two DNA vaccinations. However, this assay detected responses to the boost vaccine antigen, HPV18, as well as to HPV16, suggesting that it may be a more sensitive indicator of immunogenicity in the blood.

### Example 3

Tissue CD8+ T cell infiltrates in the target lesion increase after vaccination.

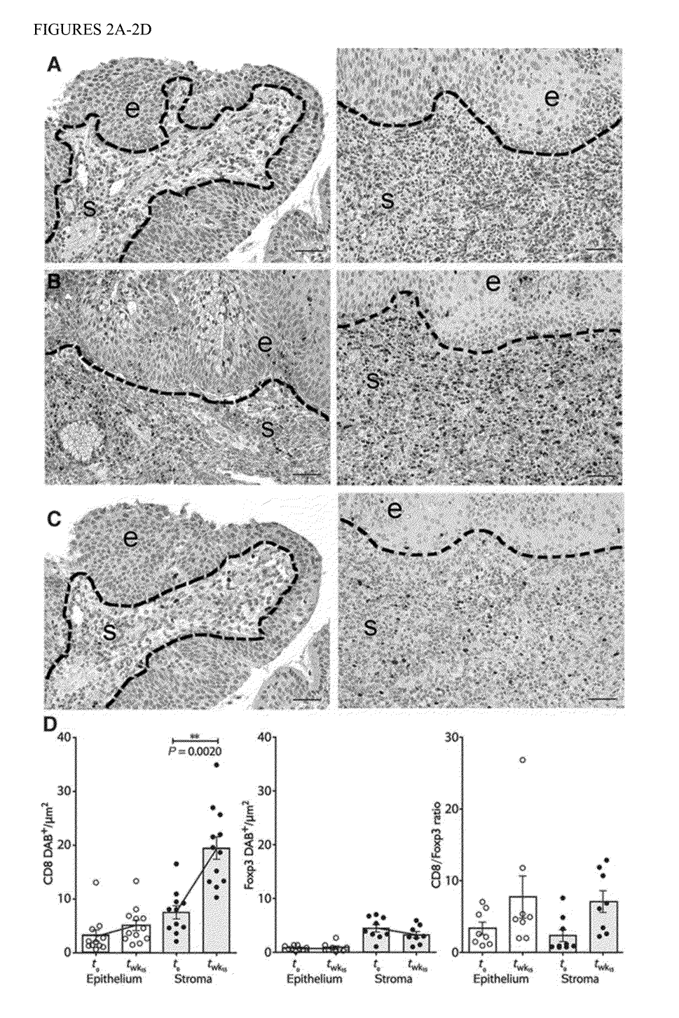

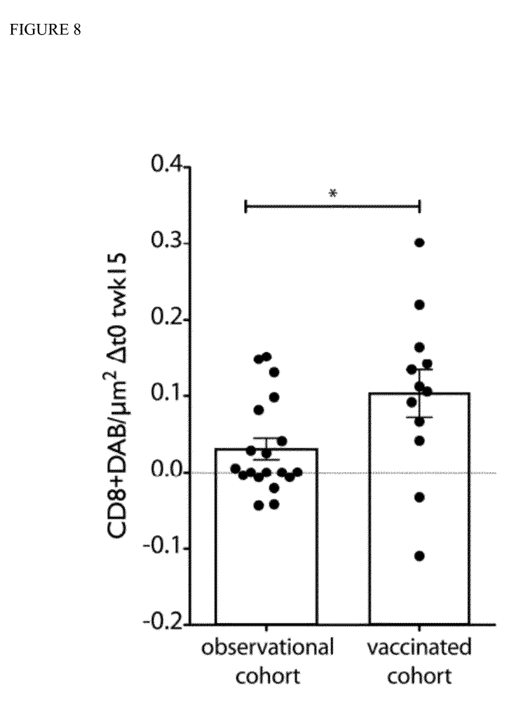

In unvaccinated persistent CIN2/3, we and others have found that tissue T cells are relatively restricted to the stromal compartment immediately subjacent to dysplastic epithelium (11, 14). This pattern of T cell distribution in unvaccinated lesions is consistent with non-antigen-specific recruitment to virally infected mucosa via a chemokine gradient. In contrast, in the post vaccination tissue specimens, the intensity of CD8+ T cell infiltration into lesions increased markedly both in the dysplastic epithelium and in the underlying stromal compartment (P=0.0020) (FIG. 2). These infiltrates were localized in foci of residual dysplasia, not in immediately adjacent normal mucosa. Within-subject increases in tissue CD8+ T cells were significantly greater than the increases we have reported previously in unvaccinated subjects followed over the same time frame (P=0.0300, FIG. 8) (11). These infiltrates included increased absolute numbers of Tbet+cells, suggestive of an effector T cell response. Intraepithelial CD8+ infiltrates were associated with histologic features of apoptosis in lesional epithelial cells. In contrast, the intensity of Foxp3+ infiltrates did not change significantly, resulting in an increased ratio of effector to Foxp3+ cells (P=0.0488).

To explore the association between the intensity of tissue T cell infiltrates and immune responses in the blood, we calculated the Pearson correlations between lesional epithelial and stromal CD8 infiltrates before and after vaccination, and peripheral blood immune responses to HPV16 E6 and E7 at baseline (before vaccination), at 8 weeks (T8), at the time of resection at week 15 (T15), and postoperatively at week 19 (T19). We found a strong association between intraepithelial CD8 infiltrates at baseline (T0) and the magnitude of T cell response to E6 in the blood after vaccination, at week 15 (r=0.742, P=0.0057) and at week 19 (r=0.751, P=0.0049). These comparisons also identified a strong correlation between the intensity of lesional stromal CD8 infiltrates at baseline and peripheral blood T cell response to E7 at week 19 (r=0.755, P=0.0045). Finally, in subjects who had foci of residual disease at week 15, we found that peripheral blood responses to E6 at weeks 15 and 19 correlated with increased intraepithelial CD8 infiltrates compared to baseline (week 15: r=0.788, P=0.0023; week 19: r=0.76, P=0.004). These findings suggest that detectable peripheral blood responses to vaccination in the setting of established pre invasive disease may reflect potentially effective, endogenous priming at the site of the lesion.

### Example 4

In vaccinated patients, the cervix is infiltrated by activated effector memory T cells with potent effector functions.

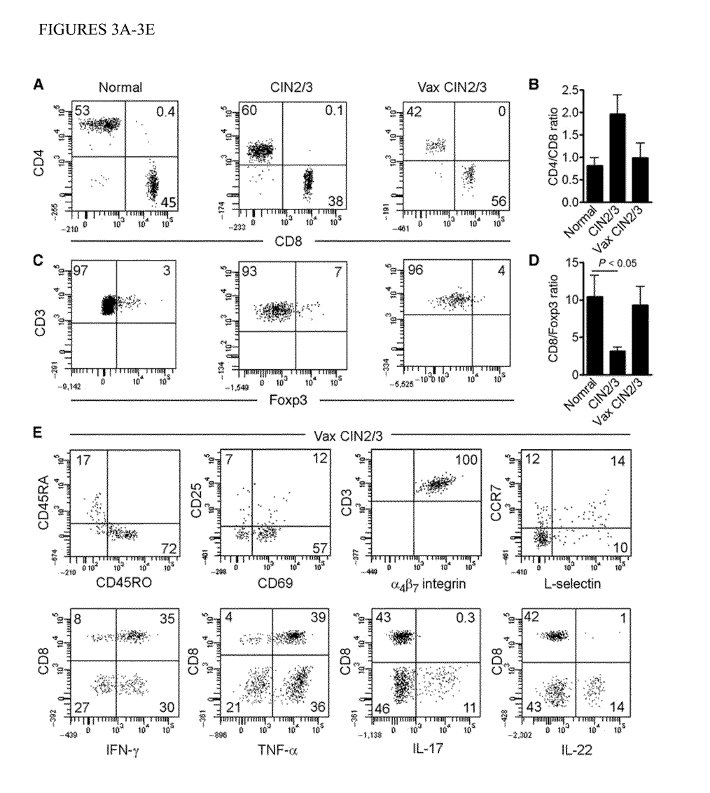

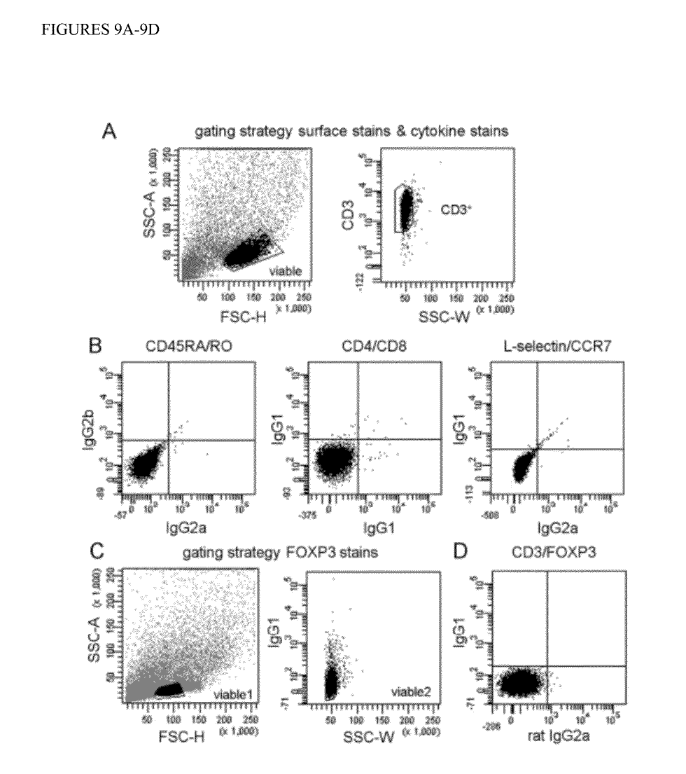

We used flow cytometry phenotyping to compare the frequencies of T cell subsets isolated from fresh tissue explants as described previously (15), in explants from normal cervical mucosa, from unvaccinated HPV16+ persistent CIN2/3, and from vaccinated HPV16+ CIN2/3. The great majority of tissue T cells in all three clinical settings had an antigen-experienced phenotype (CD3+CD45RO+) (FIG. 3 and FIG. 9). Using this method, we found differences in the ratio of effector (CD8) to regulatory T (Treg) cells in these specimens that were congruent with the image analysis data. The ratio of CD8/Treg cells was lower in vaccine-naïve persistent dysplasia, compared to normal tissue, and this ratio was reversed in post-vaccination resections. Cervical tissue T cells isolated from vaccinated subjects had an activated (CD69+), effector memory (CCR7-/L-selectin-) phenotype, and produced TH1 cytokines IFN-γ and tumor necrosis factor-α(TNF-α). All expressed the α4β7 homing integrin.

### Example 5

Tissue T cells in vaccinated subjects form tertiary lymphoid structures and show characteristics associated with in vivo activation via T cell receptor engagement with cognate antigen.

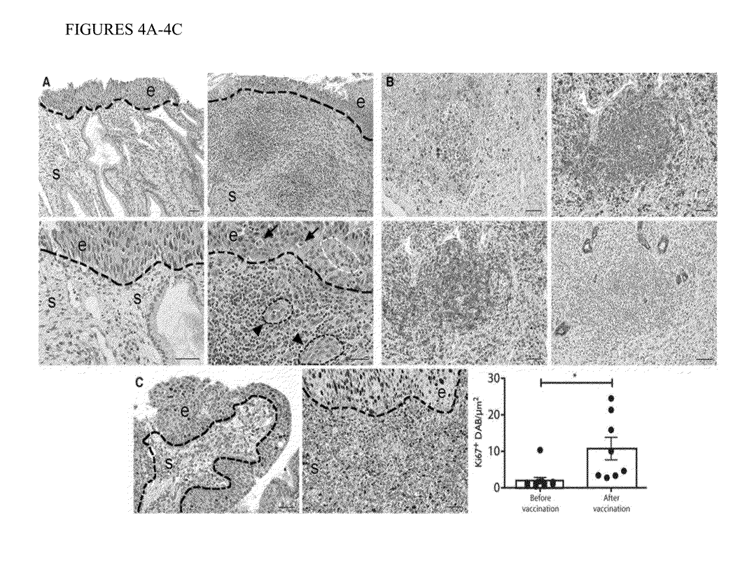

In many human solid tumors, the presence of ectopic tertiary lymphoid structures (TLSs) is associated with favorable prognosis (16-18). We found that post-vaccination changes in target lesions not only included increased intensity of CD8+ T cell infiltrates in foci of residual dysplastic epithelium but also were organized in distinct lymphoid aggregates, which included, in many cases, germinal centers (FIG. 4). In unvaccinated, persistent CIN2/3, the tissue lymphoid infiltrates localized in the stroma subjacent to dysplastic epithelium are diffuse and are not commonly organized into TLSs. In the post-vaccination tissue sections, the TLSs, similar to lymph nodes, were associated with high endothelial venules, structures that mediate lymphocyte recruitment, and are also associated with favorable prognosis (19).

The intensity and pattern of tissue CD8⁺ infiltrates also suggested activation via recognition of cognate antigen. Quantitative image analyses comparing pre- and post-vaccination lesional tissue in individual subjects showed that, after vaccination, tissue T cells had strongly increased expression of Ki67 (P=0.0062), a phenotype of recent proliferation as a consequence of activation by T cell receptor (TCR) engagement (20) (FIG. 4), providing indirect evidence of activation via cognate antigen.

### Example 6

Histologic changes in lesional stroma in vaccinated subjects are associated with a molecular signature of immune activation in both the stromal and epithelial compartments.

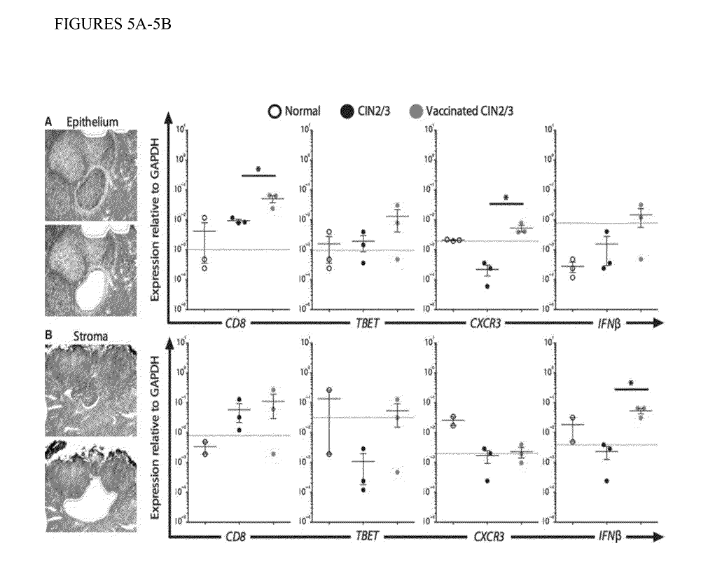

To assess the functional polarization of tissue T cells in post-vaccination tissue lymphoid aggregates, we laser capture microdissected epithelium and subjacent stroma from tissue sections from normal cervical mucosa, from unvaccinated dysplastic mucosa, and from vaccinated subjects who had residual dysplasia. Targeted analyses of genes associated with immune cell phenotype (CD8β, CD4, Foxp3, and CCR7), activation (CD25, CD69, CD137, and CXCR3), polarization (Tbet, GATA3, Foxp3, RORγt, and BCL6), and function (PRF1, GZMB, IFN-γ, and IFN-β) were carried out on laser capture microdissected epithelium and the immediately subjacent stroma from normal cervix, unvaccinated HPV16+CIN3, and from post-vaccination HPV16+CIN2/3 sections containing TLSs (FIG. 5). In vaccinated subjects, although this strategy resulted in stroma that was enriched for TLSs, the micro-dissected material also contained admixed stromal elements, including fibroblasts, macrophages, and vascular structures.

The relative expression of transcripts for CD8 and CD4 was internally congruent with quantitative IHC data. CD8 mRNA was increased in unvaccinated persistent CIN2/3 compared to normal mucosa and was even greater in vaccinated subjects. CD4 mRNA transcripts increased slightly in unvaccinated lesions compared to normal tissue and did not change significantly in vaccinated subjects. The consistency between groups for the intensity of infiltrates and the expression of transcripts suggested that the microdissections were accurate and that the quality of material was sufficient to draw conclusions from measures of other genes. In the stroma subjacent to persistent CIN2/3 from subjects in the unvaccinated cohort, we found down-regulated expression of genes associated with immune cell activation (CXCR3), T_{H}1 polarization such as Tbet (TBX21), and antiviral activity (IFN-γ and IFN-β). These findings were consistent with our previously published quantitative IHC data demonstrating increased CD8⁺ infiltrates that were nonetheless restricted to lesional stroma in unvaccinated subjects (11). In contrast, in the post-vaccination tissue sections that contained TLSs, the molecular signature suggested immune cell activation (CXCR3) and T_{H}1 polarization (TBX21, IFN-γ, and IFN-β). Similar to the CD8 and CD4 data, the changes in Tbet and Foxp3 were internally congruent with our quantitative image analyses of IHC in the same set of specimens.

We found evidence of a post-vaccination immunologic effect not only in stromal tissue but also in the overlying dysplastic epithelium. Specifically, gene transcripts for CD8β, TBX21, IFNγ, IFN-β, and CXCR3 were up-regulated in post-vaccination dysplastic epithelium compared to unvaccinated lesions. These image analysis-guided gene expression data suggest that by 7 weeks after vaccination, the epithelial compartment of dysplastic lesions is infiltrated with effector T cells that are activated and proliferating and could mediate an effect against epithelial targets expressing HPV genes.

### Example 7

After vaccination, clonally expanded TCRs are disproportionately represented in mucosal tissue compared to the blood.

To assess the repertoire of peripheral blood and tissue T cells, we determined the amino acid sequences in the hypervariable complementarity-determining region 3 (CDR3) of the TCR β chain (TCRB) in a subset of subject-matched tissue and peripheral blood T cells by high-throughput sequencing. Genomic DNA was extracted from peripheral blood T lymphocytes and cervical tissue from three subjects whose post vaccination resection specimens had an activated immune signature in the residual lesional mucosa and from an unvaccinated subject. Amplification and sequencing of TCRB CDR3 regions was carried out on the ImmunoSEQ platform at Adaptive Biotechnologies as previously described (21).

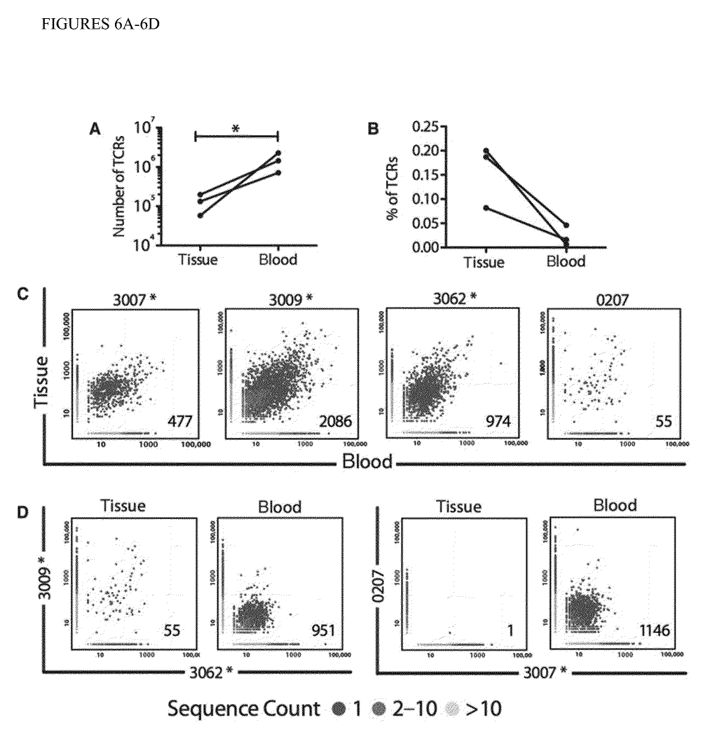

Several measures suggested that the tissue T cells were not composed of transudate from the blood but rather reflected a process of selection, presumably mediated at least in part by tissue-localized antigens. First, the diversity of the T cell clonal repertoire in each specimen was estimated using the empirical Bayesian method described previously by Daley and Smith (21, 22). The diversity of the TCR repertoire in the tissue was highly restricted compared to the blood (FIG. 6A). We then compared the frequencies of clonally expanded TCR CDR3 sequences and the degree of overlap (that is, shared TCR sequences) in tissue and blood T cells. Although many of the clonally expanded TCRs detected in vaccinated tissue could also be found in subject-matched peripheral blood, the shared sequences comprised a greater percentage of the repertoire in the tissue compared to the blood (FIG. 6B). Tissue-peripheral blood paired specimens from vaccinated subjects also had significantly greater overlap of shared TCRs than unvaccinated persistent HPV16+ CIN2/3 (FIG. 6C). Indeed, many TCRs that either were detectable with very low frequency or were undetectable in the blood were highly expanded in the tissue (data not shown). Conversely, many clonally expanded TCRs in the blood were not detectable in subject-matched tissue.

Although the samples were too small to allow assessment of the specificity of the tissue T cells, we reasoned that if the lesion-localized T cell activation and proliferation was the result of stimulation by the same lesion-associated antigens, then we might find shared TCR sequences between vaccinated subjects whose residual lesions contained stromal TLSs and who shared HLA alleles. Indeed, we found shared TCR gene usage between tissue T cells of vaccinated subjects who shared HLA alleles (FIG. 6D and Table 3) as well as in the blood. In contrast, when we compared TCR sequences in an unvaccinated and a vaccinated subject who shared HLA A201, we identified overlap only in the blood. Together, these findings suggest that the T cell repertoire in the post-vaccination tissue is both local and specific.

TABLE 3
Shared tissue TCR between two vaccinated subjects.

| | Percent of total | | | |
|---|---|---|---|---|
| Amino acid | 3009SA | 3062MH | V family | J gene |
| CASSDSSGSTDTQYF (SEQ ID NO: 1) | 6.03729 | 0.471434 | 6 | TRBJ2-3 |
| CASSQGGLAGVNEQFF (SEQ ID NO: 2) | 2.02448 | 6.026706 | 3 | TRBJ2-1 |
| CASSDSTSGSNEQFF (SEQ ID NO: 3) | 2.02448 | 1.083023 | 6 | TRBJ2-1 |
| CASSQVEETQYF (SEQ ID NO: 4) | 0.57842 | 0.598848 | 3 | TRBJ2-5 |
| CASSLGPHNEQFF (SEQ ID NO: 5) | 0.76822 | 0.586107 | 7 | TRBJ2-1 |
| CASSADTRYNEQFF (SEQ ID NO: 6) | 0.61457 | 0.789970 | 2 | TRBJ2-1 |
| CASSLNSYEQYF (SEQ ID NO: 7) | 0.30729 | 0.420468 | 5 | TRBJ2-7 |
| CASSLAGGYTF (SEQ ID NO: 8) | 0.35248 | 0.293053 | 12 | TRBJ1-2 |
| CASSLLARGTDTQYF (SEQ ID NO: 9) | 0.19883 | 0.458692 | 5 | TRBJ2-3 |
| CASSYRGTDTQYF (SEQ ID NO: 10) | 0.10845 | 0.191122 | 6 | TRBJ2-3 |
| CASSVGTGNQPQHF (SEQ ID NO: 11) | 0.06326 | 0.356761 | 9 | TRBJ1-5 |
| CSARDRTSGSYEQYF (SEQ ID NO: 12) | 0.14461 | 0.152897 | 20 | TRBJ2-7 |
| CATSRDRAADTQYF (SEQ ID NO: 13) | 0.09038 | 0.203863 | 15 | TRBJ2-3 |
| CASSPGQGAETQYF (SEQ ID NO: 14) | 0.15364 | 0.063707 | 7 | TRBJ2-5 |
| CASSLAADTQYF (SEQ ID NO: 15) | 0.08134 | 0.140156 | 5 | TRBJ2-3 |

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### REFERENCES

1. C. de Martel, et al., Global burden of cancers attributable to infections in 2008: A review and synthetic analysis. Lancet Oncol. 13, 607-615 (2012).
2. A. Jemal, et al., Annual Report to the Nation on the Status of Cancer, 1975-2009, featuring the burden and trends in human papillomavirus (HPV)-associated cancers and HPV vaccination coverage levels. J. Natl. Cancer Inst. 105, 175-201 (2013).
3. R. L. Winer, et al., Risk of female human papillomavirus acquisition associated with first male sex partner. J. Infect. Dis. 197, 279-282 (2008).
4. Centers for Disease Control and Prevention (CDC), Human papillomavirus vaccination coverage among adolescent girls, 2007-2012, and postlicensure vaccine safety monitoring, 2006-2013—United States. MMWR Morb. Mortal. Wkly. Rep. 62, 591-595 (2013).
5. J. Hudson, M. et al , Immortalization and altered differentiation of human keratinocytes in vitro by the E6 and E7 open reading frames of human papillomavirus type 18. J. Virol. 64, 519-526 (1990).
6. B. Werness, et al., Association of human papillomavirus types 16 and 18 E6 proteins with p53. Science 248, 76-79 (1990).
7. J. Melnikow, et al., Natural history of cervical squamous intraepithelial lesions: A meta-analysis. Obstet. Gynecol. 92, 727-735 (1998).
8. C. L. Trimble, et al., Spontaneous regression of high-grade cervical dysplasia: Effects of human papillomavirus type and HLA phenotype. Clin. Cancer Res. 11, 4717-4723 (2005).
9. M. Nakagawa, et al., Persistence of human papillomavirus type 16 infection is associated with lack of cytotoxic T lymphocyte response to the E6 antigens. J. Infect. Dis. 182, 595-598 (2000).
10. C. L. Trimble, et al., Naturally occurring systemic immune responses to HPV antigens do not predict regression of CIN2/3. Cancer Immunol. Immunother. 59, 799-803 (2010).
11. C. L. Trimble, et al., Human papillomavirus 16-associated cervical intraepithelial neoplasia in humans excludes CD8 T cells from dysplastic epithelium. J. Immunol. 185, 7107-7114 (2010).
12. S. Dubey, et al., Detection of HIV vaccine-induced cell-mediated immunity in HIV-seronegative clinical trial participants using an optimized and validated enzyme linked immunospot assay. J. Acquir. Immune Defic. Syndr. 45, 20-27 (2007).
13. S. P. Buchbinder, et al.; Step Study Protocol Team, Efficacy assessment of a cell-mediated immunity HIV-1 vaccine (the Step Study): A double-blind, randomised, placebo-controlled, test-of-concept trial. Lancet 372, 1881-1893 (2008).
14. A. Kobayashi, et al., Functional attributes of mucosa! immunity in cervical intraepithelial neoplasia and effects of HIV infection. Cancer Res. 64, 6766-6774 (2004).
15. R. A. Clark, et al., A novel method for the isolation of skin resident T cells from normal and diseased human skin. J. Invest. Dermatol. 126, 1059-1070 (2006).
16. M. C. Dieu-Nosjean, et al., Long-term survival for patients with non-small-cell lung cancer with intratumoral lymphoid structures. J. Clin. Oncol. 26, 4410-4417 (2008).
17. J. L. Messina, et al., 12-Chemokine gene signature identifies lymph node-like structures in melanoma: Potential for patient selection for immunotherapy? Sci. Rep. 2, 765 (2012).
18. D. Coppola, et al., Unique ectopic lymph node-like structures present in human primary colorectal carcinoma are identified by immune gene array profiling. Am. J. Pathol. 179, 37-45 (2011).
19. L. Martinet, et al., Human solid tumors contain high endothelial venules: Association with T- and B-lymphocyte infiltration and favorable prognosis in breast cancer. Cancer Res. 71, 5678-5687 (2011).
20. T. Scholzen, J. Gerdes, The Ki-67 protein: From the known and the unknown. J. Cell. Physiol. 182, 311-322 (2000).
21. H. S. Robins, et al., Comprehensive assessment of T-cell receptor b-chain diversity in ab T cells. Blood 114, 4099-4107 (2009).
22. T. Daley, A. D. Smith, Predicting the molecular complexity of sequencing libraries. Nat. Methods 10, 325-327 (2013).
23. M. van Seters, et al., Is the assumed natural history of vulvar intraepithelial neoplasia III based on enough evidence? A systematic review of 3322 published patients. Gynecol. Oncol. 97, 645-651 (2005).
24. G. G. Kenter, et al., Vaccination against HPV-16 oncoproteins for vulvar intraepithelial neoplasia. N. Engl. J. Med. 361, 1838-1847 (2009).
25. S. Daayana, et al., Phase II trial of imiquimod and HPV therapeutic vaccination in patients with vulval intraepithelial neoplasia. Br. J. Cancer 102, 1129-1136 (2010).
26. F. Pagès, et al., Effector memory T cells, early metastasis, and survival in colorectal cancer. N. Engl. J. Med. 353, 2654-2666 (2005).
27. W. H. Fridman, et al., The immune contexture in human tumours: Impact on clinical outcome. Nat. Rev. Cancer 12, 298-306 (2012).
28. L. de Chaisemartin, et al., Characterization of chemokines and adhesion molecules associated with T cell presence in tertiary lymphoid structures in human lung cancer. Cancer Res. 71, 6391-6399 (2011).
29. A. Byshevskii, et al., Erythrocytes and leucocytes in realization of communication between lipid peroxidation and hemostasis. Biomed. Khim. 52, 370-377 (2006).
30. J. Galon, et al., Cancer classification using the Immunoscore: A worldwide task force. J. Transl. Med. 10, 205 (2012).
31. M. L. Bagarazzi, et al, Immunotherapy against HPV16/18 generates potent TH1 and cytotoxic cellular immune responses. Sci. Transl. Med. 4, 155ra138 (2012).
32. A. Bere, et al., Comparison of polyclonal expansion methods to improve the recovery of cervical cytobrush-derived T cells from the female genital tract of HIV-infected women. J. Immunol. Methods 354, 68-79 (2010).
33. D. Kalamaszet al., Optimization of human T-cell expansion ex vivo using magnetic beads conjugated with anti-CD3 and anti-CD28 antibodies. J. Immunother. 27, 405-418 (2004).
34. V. Corbière, et al., Antigen spreading contributes to MAGE vaccination-induced regression of melanoma metastases. Cancer Res. 71, 1253-1262 (2011).
35. A. Cipponi, et al., Neogenesis of lymphoid structures and antibody responses occur in human melanoma metastases. Cancer Res. 72, 3997-4007 (2012).
36. L. Ding, et al., Somatic mutations affect key pathways in lung adenocarcinoma. Nature 455,1069-1075 (2008).
37. C. L. Trimble, et al., A phase I trial of a human papillomavirus DNA vaccine for HPV16+ cervical intraepithelial neoplasia 2/3. Clin. Cancer Res. 15, 361-367 (2009).
38. L. K. Borysiewicz, et al., A recombinant vaccinia virus encoding human papillomavirus types 16 and 18, E6 and E7 proteins as immunotherapy for cervical cancer. Lancet 347, 1523-1527 (1996).
39. A. M. Kaufmann, et al., Safety and immunogenicity of TA-HPV, a recombinant vaccinia virus expressing modified human papillomavirus (HPV)-16 and HPV-18 E6 and E7 genes, in women with progressive cervical cancer. Clin. Cancer Res. 8, 3676-3685 (2002).
40. P. J. Baldwin, et al., Vaccinia-expressed human papillomavirus 16 and 18 E6 and E7 as a therapeutic vaccination for vulval and vaginal intraepithelial neoplasia. Clin. Cancer Res. 9, 5205-5213 (2003).
41. E. J. Davidson, et al , Immunological and clinical responses in women with vulval intraepithelial neoplasia vaccinated with a vaccinia virus encoding human papillomavirus 16/18 oncoproteins. Cancer Res. 63, 6032-6041 (2003).
42. J. D. Boyer, et al., SIV DNA vaccine co-administered with IL-12 expression plasmid enhances CD8 SIV cellular immune responses in cynomolgus macaques. J. Med. Primatol. 34, 262-270 (2005).
43. S. Janetzki, et al., Elispot Proficiency Panel of the CVC Immune Assay Working Group, Results and harmonization guidelines from two large-scale international Elispot proficiency panels conducted by the Cancer Vaccine Consortium (CVC/SVI). Cancer Immunol. Immunother. 57, 303-315 (2008).
44. J. Yan, et al., Induction of antitumor immunity in vivo following delivery of a novel HPV-16 DNA vaccine encoding an E6/E7 fusion antigen. Vaccine 27, 431-440 (2009).
45. J. Yan, et al., Cellular immunity induced by a novel HPV18 DNA vaccine encoding an E6/E7 fusion consensus protein in mice and rhesus macaques. Vaccine 26, 5210-5215 (2008).
46. M. P. Morrow, et al., IL-28B/IFN-13 drives granzyme B loading and significantly increases CTL killing activity in macaques. Mol. Ther. 18, 1714-1723 (2010).
47. M. Yousfi Monod, et al., IMGT/JunctionAnalysis: The first tool for the analysis of the immunoglobulin and T cell receptor complex V-J and V-D-J JUNCTIONs. Bioinformatics 20 (Suppl. 1), i379-i385 (2004).
48. R. A. Fisher, et al., The relation between the number of species and the number of individuals in a random sample of an animal population. J. Anim. Ecol. 12, 42-58 (1943).

1. A method for determining the effectiveness of an HPV vaccine regimen on a subject having a cervical intraepithelial neoplasia (CIN) comprising: a) obtaining at least one first tissue sample from the CIN of the subject; b) administering to the subject a first vaccine construct comprising HPV16 E7 antigen one or more times; c) administering to the subject a second vaccine construct comprising both E6 and E7 antigens from both HPV16 and HPV18; d) obtaining at least one or more second tissue samples from the CIN of the subject about 4 to about 24 weeks after step c); e) measuring the amount of CD8+ T cell infiltrates in the intraepithelial tissue of the first and second CIN samples; and f) determining that the HPV vaccine regimen is effective when the amount of CD8+ T cell infiltrates in the second tissue sample is increased when compared to the amount of CD8+ T cell infiltrates in the first tissue sample.

2. The method of claim 1, wherein at step d), the at least one or more samples are obtained at 4 weeks to about 8 weeks after step c).

3. The method of claim 1, wherein at step f), when the amount of CD8+ T cell infiltrates in the second tissue sample is the same or decreased when compared to the amount of CD8+ T cell infiltrates in the first tissue sample, then the HOV vaccine regimen is determined to be ineffective.

4. The method of claim 1, further comprising: step e1) measuring the quantity of Treg cells in the intraepithelial tissue of the first and second CIN samples; and f1) determining that the HPV vaccine regimen is effective when the ratio of the quantity of CD8+ T cells/Treg cells in the second tissue sample is increased when compared to the ratio of the quantity of CD8+ T cells/Treg in the first tissue sample.

5. The method of claim 4, wherein at step f1), when the ratio of the quantity of CD8+ T cells/Treg cells in the second tissue sample is the same or decreased when compared to the ratio of the quantity of CD8+ T cells/Treg cells in the first tissue sample, then the HPV vaccine regimen is determined to be ineffective.

6. A method for treating a subject having a cervical intraepithelial neoplasia (CIN) comprising: a) obtaining at least one first tissue sample from the CIN of the subject; b) administering to the subject a first vaccine construct comprising HPV16 E7 antigen one or more times; c) administering to the subject a second vaccine construct comprising both E6 and E7 antigens from both HPV16 and HPV18; d) obtaining at least one or more second tissue samples from the CIN of the subject about 4 to about 24 weeks after step c); e) measuring the amount of CD8+ T cell infiltrates in the intraepithelial tissue of the first and second CIN samples; and f) determining that the HPV treatment is effective when the amount of CD8+ T cell infiltrates in the second tissue sample is increased when compared to the amount of CD8+ T cell infiltrates in the first tissue sample.

7. The method of claim 6, wherein at step d), the at least one or more samples are obtained at 4 weeks to about 8 weeks after step c).

8. The method of claim 6, further comprising: step e1) measuring the quantity of Treg cells in the intraepithelial tissue of the first and second CIN samples; and f1) determining that the HPV vaccine regimen is effective when the ratio of the quantity of CD8+ T cells/Treg cells in the second tissue sample is increased when compared to the ratio of the quantity of CD8+ T cells/Treg in the first tissue sample.

9. A method for determining the effectiveness of an HPV vaccine regimen with an adjuvant on a subject having a cervical intraepithelial neoplasia (CIN) comprising:
a) obtaining at least one first tissue sample from the CIN of the subject;
b) administering to the subject an effective amount of a first vaccine construct comprising HPV16 E7 antigen one or more times;
c) administering to the subject an effective amount of a second vaccine construct comprising both E6 and E7 antigens from both HPV16 and HPV18, and an effective amount of an adjuvant;
d) obtaining at least one or more second tissue samples from the CIN of the subject about 4 to about 24 weeks after step c);
e) measuring the amount of CD8+ T cell infiltrates in the intraepithelial tissue of the first and second CIN samples; and
f) determining that the HPV treatment is effective when the amount of CD8+ T cell infiltrates in the second tissue sample is increased when compared to the amount of CD8+ T cell infiltrates in the first tissue sample.

10. A method for treating a subject having a cervical intraepithelial neoplasia (CIN) comprising:
a) obtaining at least one first tissue sample from the CIN of the subject;
b) administering to the subject an effective amount of a first vaccine construct comprising HPV16 E7 antigen one or more times;
c) administering to the subject an effective amount of a second vaccine construct comprising both E6 and E7 antigens from both HPV16 and HPV18, and an effective amount of an adjuvant;
d) obtaining at least one or more second tissue samples from the CIN of the subject about 4 to about 24 weeks after step c);
e) measuring the amount of CD8+ T cell infiltrates in the intraepithelial tissue of the first and second CIN samples; and
f) determining that the HPV treatment is effective when the amount of CD8+ T cell infiltrates in the second tissue sample is increased when compared to the amount of CD8+ T cell infiltrates in the first tissue sample.

11. The method of claim 9, wherein at step d), the at least one or more samples are obtained at 4 weeks to about 8 weeks after step c).

12. The method of claim 10, wherein at step d), the at least one or more samples are obtained at 4 weeks to about 8 weeks after step c).

13. The method of claim 9, wherein at step f), when the quantity of CD8+ T cells in the second tissue sample is the same or decreased when compared to the quantity of CD8+ T cells in the first tissue sample, then the HOV vaccine regimen is determined to be ineffective.

14. The method of claim 10, wherein at step f), when the quantity of CD8+ T cells in the second tissue sample is the same or decreased when compared to the quantity of CD8+ T cells in the first tissue sample, then the HOV vaccine regimen is determined to be ineffective.

15. The method of claim 9, further comprising: step e1) measuring the quantity of Treg cells in the intraepithelial tissue of the first and second CIN samples; and f1) determining that the HPV vaccine regimen is effective when the ratio of the quantity of CD8+ T cells/Treg cells in the second tissue sample is increased when compared to the ratio of the quantity of CD8+ T cells/Treg in the first tissue sample.

16. The method of claim 10, further comprising: step e1) measuring the quantity of Treg cells in the intraepithelial tissue of the first and second CIN samples; and f1) determining that the HPV vaccine regimen is effective when the ratio of the quantity of CD8+ T cells/Treg cells in the second tissue sample is increased when compared to the ratio of the quantity of CD8+ T cells/Treg in the first tissue sample.

17. The method of claim 15, wherein at step f1), when the ratio of the quantity of CD8+ T cells/Treg cells in the second tissue sample is the same or decreased when compared to the ratio of the quantity of CD8+ T cells/Treg cells in the first tissue sample, then the HPV vaccine regimen is determined to be ineffective.


SEQUENCE LISTING (15 sequences)

SEQ ID NO: 1 - protein, 15 aa, Homo sapiens
CASSDSSGSTDTQYF

SEQ ID NO: 2 - protein, 16 aa, Homo sapiens
CASSQGGLAGVNEQFF

SEQ ID NO: 3 - protein, 15 aa, Homo sapiens
CASSDSTSGSNEQFF

SEQ ID NO: 4 - protein, 12 aa, Homo sapiens
CASSQVEETQYF

SEQ ID NO: 5 - protein, 13 aa, Homo sapiens
CASSLGPHNEQFF

SEQ ID NO: 6 - protein, 14 aa, Homo sapiens
CASSADTRYNEQFF

SEQ ID NO: 7 - protein, 12 aa, Homo sapiens
CASSLNSYEQYF

SEQ ID NO: 8 - protein, 11 aa, Homo sapiens
CASSLAGGYTF

SEQ ID NO: 9 - protein, 15 aa, Homo sapiens
CASSLLARGTDTQYF

SEQ ID NO: 10 - protein, 13 aa, Homo sapiens
CASSYRGTDTQYF

SEQ ID NO: 11 - protein, 14 aa, Homo sapiens
CASSVGTGNQPQHF

SEQ ID NO: 12 - protein, 15 aa, Homo sapiens
CSARDRTSGSYEQYF

SEQ ID NO: 13 - protein, 14 aa, Homo sapiens
CATSRDRAADTQYF

SEQ ID NO: 14 - protein, 14 aa, Homo sapiens
CASSPGQGAETQYF

SEQ ID NO: 15 - protein, 12 aa, Homo sapiens
CASSLAADTQYF